(19) United States
(12) Patent Application Publication
(10) Pub. No.: US 2026/0028371 A1
(43) Pub. Date: Jan. 29, 2026

(54) HIGH PERFORMANCE AND ENVIRONMENTALLY COMPATIBLE FILMS, FIBERS, AND MEMBRANES WITH BUNDLEMER BUILDING BLOCKS

(71) Applicant: University of Delaware, Newark, DE (US)

(72) Inventors: Darrin J. Pochan, Landenberg, PA (US); Christopher Kloxin, Landenberg, PA (US); Amanda McCahill, Newark, DE (US); Yi Shi, Newark, DE (US); Tianren Zhang, Princeton, NJ (US); Yao Tang, Newark, DE (US); Albree Weisen, Newark, DE (US); Jeffery Saven, Philadelphia, PA (US)

(21) Appl. No.: 19/284,152

(22) Filed: Jul. 29, 2025

Related U.S. Application Data
(60) Provisional application No. 63/676,484, filed on Jul. 29, 2024.

(51) Int. Cl. C07K 1/107; C07K 19/00; C08F 2/48

(52) U.S. Cl. CPC C07K 1/1077; C07K 19/00; C08F 2/48

(57) ABSTRACT

Disclosed herein are to peptide bundlemers that possess one or more hydrophobic groups on the surface that are capable of assembling into lattice nanostructures, amorphous networks and liquid crystalline structures in solution. Methods of creating these structures and articles formed from these structures are also disclosed.

### CROSS-REFERENCE TO RELATED APPLICATION

This application claims priority to U.S. Provisional Patent Application No. 63/676,484 filed on Jul. 29, 2024, the content of which is incorporated herein by reference in its entirety for all purposes.

### STATEMENT REGARDING FEDERALLY SPONSORED RESEARCH

This invention was made with government support under Grant Nos. DE-SC0019355 and DE-SC0019282 awarded by the US Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 28, 2025 is named 2101715-001315.xml and is 23,519 bytes in size.

### FIELD

The present disclosure relates to peptide bundlemers capable of assembling into lattice nanostructures, amorphous networks and liquid crystalline structures in solution. Methods of creating these structures and articles formed from these structures are also disclosed.

### BACKGROUND

The ability to design, synthesize, and characterize exact nanoscale architectures and polymer networks inspired by natural proteins is an ongoing challenge in the materials field. Indeed, though polymer networks have been studied extensively (e.g., those containing covalently crosslinked networks, supramolecular networks, covalent adaptable networks, and stimuli responsive polymer networks), all polymer networks face the same fundamental challenge: molecular control of physical/covalent crosslinks.

Many polymers make inhomogeneous networks where defects often dictate mechanical behavior, and precise control of crosslinking at the molecular level is a challenge that researchers are addressing. Complementary to synthetic polymers, biological molecules (e.g., DNA, proteins, and peptides) provide many unique molecular attributes in the formation of synthetic materials. Proteins, for example, offer sequence specificity (e.g., in their primary structure/amino acid sequence) that can enable templated display of functional groups. Proteins also offer specific secondary structures (e.g., beta sheets, alpha helices, random coils) and can exhibit intermolecular structures (e.g., beta sheet peptide fibrils, multimer proteins, peptide and protein coiled coils). The combination of the above biomolecular attributes can provide the opportunity to purposefully use a specific, spatial display of chemical functionality to dictate formation of designed nanostructures in synthetic materials.

Thus, to address the foregoing issues surrounding polymer network design, the present disclosure provides novel tetrameric coiled-coil peptide bundlemer structures that allow for purposeful spatial display of chemical functional groups to form desired intra- and interbundle crosslinking interactions, therefore providing fine control over the formation of polymer networks and their ultimate mechanical properties.

### SUMMARY

One aspect of the present disclosure is a bundlemer including (i.e., comprising) four peptides, each peptide possessing a first amino acid sequence (1^{st}), a second amino acid sequence (2^{nd}), a third amino acid sequence (3^{rd}) and a fourth amino acid sequence (4^{th}), wherein the 1^{st}, 2^{nd}, 3^{rd} and 4^{th} amino acid sequence possess a heptadic peptide sequence abcdefg and are arranged as follows:

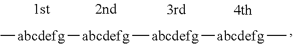

wherein (i) the 1^{st}, 2^{nd}, 3^{rd} and 4^{th} amino acid sequences together form an alpha-helical structure; (ii) the a, d, and g positions form a hydrophobic core of the tetrameric coiled-coil bundlemer particle, wherein: the a position of the 1^{st} amino acid sequence is aspartic acid (D) and the a position of the 2^{nd}, 3^{rd} and 4^{th} amino acid sequences is alanine (A), the d position of the 1^{st}, 2^{nd}, 3^{rd} and 4^{th} amino acid sequences is isoleucine (I), and the g position of the 1^{st}, 2^{nd}, and 3^{rd} amino acid sequence is methionine (M) and the g position of the 4^{th} amino acid sequence is either methionine (M) or glutamic acid (E); (iii) each b, c, e, and f is an amino acid exposed on a surface of the bundlemer particle structure; and (iv) at least one of the b, c, e, and f positions of the 2^{nd} and/or 3^{rd} amino acid sequence is selected from a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

Another aspect of the present disclosure is a second bundlemer including four peptides, each peptide possessing a first amino acid sequence (1^{st}), a second amino acid sequence (2^{nd}), a third amino acid sequence (3^{rd}) and a fourth amino acid sequence (4^{th}), wherein the 1^{st}, 2^{nd}, 3^{rd} and 4^{th} amino acid sequence possess a heptadic peptide sequence abcdefg and are arranged as follows:

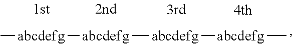

wherein (i) the 1^{st}, 2^{nd}, 3^{rd} and 4^{th} amino acid sequences together form an alpha-helical structure; (ii) the a, d, and g positions form a hydrophobic core of the tetrameric bundlemer particle, the a position of the 2^{nd}, 3^{rd} and 4^{th} amino acid sequences is alanine (A), the d position of the 1^{st}, 2^{nd}, 3^{rd} and 4^{th} amino acid sequences is isoleucine (I), and the g position of the 1^{st}, 2^{nd}, and 3^{rd} amino acid sequences is methionine (M); (iii) each b, c, e, and f is an amino acid exposed on a surface of the bundlemer particle structure; and (iv) at least one of the b, c, e, and f positions of the 2^{nd} and/or 3^{rd} amino acid sequence is selected from the group consisting of a modified lysine amino acid possessing an allyloxy carbonyl moiety, a modified lysine amino acid possessing an alkyne moiety, a modified lysine amino acid possessing a furan moiety, phenylalanine (F), tyrosine (Y) and tryptophan (W).

Aspects of the present disclosure also include lattice nanostructures, amorphous networks and liquid crystalline structures formed from the bundlemers disclosed herein.

Other aspects of the present disclosure include methods for forming the lattice nanostructures, amorphous networks and liquid crystalline structures disclosed herein.

Another aspect of the present disclosure is articles formed from the lattice nanostructures, amorphous networks or liquid crystalline structures disclosed herein.

### BRIEF DESCRIPTION OF THE DRAWINGS

Other features and advantages of the compositions, devices and methods disclosed herein will be apparent to those skilled in the art reading the following detailed description in conjugation with the exemplary embodiments illustrated in the drawings, wherein:

FIG. 1 depicts a self-assembly schematic for a lattice formation from individual, exemplary CG bundlemers. Different bundlemer particles are represented by differently colored beads for clarity.

FIG. 2 depicts amino acid sequences for exemplary modified bundlemers. The heptad repeat (abcdefg) heading indicates the positions of amino acids in the coiled coil design where positions a, d and g form the hydrophobic core. Positions b, c, e, and f are exposed on the surface of the bundlemer (KAlloc is shown as bold K; cysteine is shown as bold C). 3D rendering of the surface of the 4B+4 sequence is also shown with an accompanying cylinder schematic that highlights the shape and orientation of the constituent four helical peptides.

FIG. 3 depicts cylinder schematics for sequences 1A1C, 2A1C and 2A2C with reactive sites highlighted.

FIG. 4 depicts oscillatory rheometry time-sweep results for sequences 1A1C, 2A1C, and 2A2C at 0.25 g/mL, 0.50 g/mL, and 0.75 g/mL hydrated films. Storage (elastic) moduli, G′, are monitored as a function of time in which the UV irradiation begins at time point zero. For all cases at all concentrations G′>>G″. (Loss moduli, G″).

FIG. 5 depicts the amino acid sequences for 4B+4_2A, 2A2C and 2A2CM. Cylinder models for the modified 4B+4 sequences are also shown with reactive sites highlighted.

FIG. 6 depicts (1) oscillatory rheometry time-sweep results for 4B+4_2A at 0.25 g/mL and 0.50 g/mL showing how small the effect of alloc-alloc crosslinking is on the network properties relative to the thiol-ene reaction. Oscillatory rheometry time-sweep results for 2A2C and 2A2CM at 0.25 g/mL, 0.50 g/mL, and 0.75 g/mL (2-4) hydrated films are also shown. Storage (elastic) moduli, G′, are monitored as a function of time in which the UV irradiation begins at time point zero. For all cases at all concentrations G′>>G″.

FIG. 7 depicts amino acid sequences for 4B+4, 4B+4_2A, 4B+2 and 4B+2_2a. A 3D rendering of the surface of 4B+4 and 4B+2 are also shown, along with cylinder models of 4B+4_2A and 4B+2_2A with reactive sites highlighted.

FIG. 8 depicts TEM images of (a) a negative stain TEM of 4B+2_2A at 50 mM concentration in MilliQ, (b) a Cryo-TEM of 4B+2_2A at 50 mM concentration in MilliQ, (c) a negative stain TEM 4B+4_2A at 50 mM concentration (top left is 50 mM phosphate buffer to highlight large particles, top right/bottom are MilliQ sample to highlight small particles) and (d) a Cryo-TEM of 4B+4_2A at 50 mM concentration in 50 mM phosphate buffer.

FIG. 9 depicts the SAXS results for 4B+4_2A and 4B+2_2A at 5 wt/vol % in 50 mM phosphate buffer.

FIG. 10 depicts the atomistic display of alloc groups (spacefilling) determined via all atom simulations of an exemplary tetrahelical bundlemer (left) with the corresponding CG bundlemer model rendered as black spheres.

FIG. 11 depicts a comparison of SAXS experimental results to predicted peaks from a CG model of an exemplary lattice.

FIG. 12 depicts different viewpoints of an exemplary CG model lattice compared to cryoTEM data of 4B+2_2A in MilliQ (50 mM).

FIG. 13 depicts (A) amino acid sequences for sequence 4B+4 and sequence 4B+4_2X, where the sequences are modified at the 13^{th} and 19^{th} positions and X is the non-natural or natural amino acid replacement in 4B+4_2X (*When X=Y, then Y is changed to a Q (Gln)); (B) Structures for the non-natural amino acids; and (C) natural amino acids.

FIG. 14 depicts negative stain TEM and Cryo-TEM images of structures formed from 4B+4_2X bundlemers wherein X is Phenylalanine, Tyrosine, and a modified lysine containing an alkyne moiety. Images were captured in MilliQ (titrated to pH 7).

FIG. 15 depicts negative stain TEM and Cryo-TEM images of structures formed from 4B+4_2X bundlemers wherein X is Tryptophan and a modified lysine containing a furan moiety. Images were captured in MilliQ (titrated to pH 7).

FIG. 16 depicts SAXS data for 4B+4_2X bundlemers wherein X is Phenylalanine, Tyrosine, a modified lysine containing an alkyne moiety, Tryptophan and a modified lysine containing a furan moiety. All bundlemers were prepared at 5 w/v % in MilliQ (titrated to pH 7).

FIG. 17 depicts computational models of (A) alloc, (B) alkyne, and (C) phenylalanine modified bundlemers as monomers and dimers. Left: amino acid at positions 13 and 19 of 4B+4_2X sequence. Center: two renderings of the bundlemer monomer that differ by a rotation of 90°. Right: representative configuration of the dimer of bundlemers. In each case, the peptide backbone is represented by the ribbon. Side chains of the amino acids at the 13^{th} and 19^{th} positions are rendered as spacefilling.

FIG. 18 depicts (A) SAXS structure factors of three bundlemer variants of the 4B+4_2X sequence, wherein X is Tyrosine, Phenylalanine and a modified lysine containing an alkyne moiety (locations of features used in the fitting are indicated by vertical gray lines); (B) a model lattice determined by fitting the SAXS structure factors; and (C) Rotation (90°) of the fitted lattice in (B).

FIG. 19 depicts a hypothesized structure formed by the 4B+4_2X sequence, wherein X is tryptophan. (A) Tube schematic with spacings based on TEM measurements. Top: Single nanotube and Bottom: 2 connected nanotubes. Cross sections show the alignment of bundlemers in the outer layer of the tube. (B) Potential bundlemer packing for single and connected tubes.

FIG. 20 depicts the structures formed by the 4B+4_2X sequence, wherein X is tryptophan, in (A) negative stain TEM and (B) cryoTEM and the measured physical properties of these structures. Statistics for the measurements are found in Table 3.

FIG. 21 depicts amino acid sequences for single charge (SC) bundlemer sequences and alloc-protected lysine modified sequences. Positions a, d, and g in the heptad repeat (abcdefg) make up the hydrophobic core. K represents alloc-protected lysine.

FIG. 22 depicts (A) Small angle x-ray scattering (SAXS) for SC+6_2A (left) and SC+8_2A (right) at 5, 10, 15, and 20 w/w % in Milli-Q water; (B) Polarized optical microscopy (POM) images showing birefringence in SC+6_2A (left) and SC+8_2A (right) at 20 w/w % in Milli-Q water; and (C) CryoTEM of liquid crystal of SC+6_2A (left) and SC+8_2A (right) at 20 w/w % in Milli-Q water.

FIG. 23 depicts (A) TEM images of SC+6_2A at 5 w/w % in 1M NaCl (Top: Negative stain TEM. Bottom: Cryo-TEM); and (B) TEM images of SC+8_2A at 5 w/w % in 1M NaCl (Top: Negative stain TEM. Bottom: Cryo-TEM).

FIG. 24 depicts SAXS plots for SC+6_2A at 10 w/w % in 0.5M NaCl and SC+8_2A at 5 w/w % in 0.5M NaCl.

FIG. 25 depicts (A) Lattice packing model for SC+8_2A derived from experimental SAXS results; (B) Lattice model projection matched to cryoTEM results for SC+8_2A (Different hues are used to highlight distinct bundlemer particles in both SC+6_2A and SC+8_2A); (C) Lattice packing model for SC+6_2A derived from experimental SAXS results; and (D) Lattice model projection matched to Cryo-TEM results for SC+6_2A.

FIG. 26 depicts Negative Stain TEM of (A) SC+8_2A at 5 w/w % in 1 M NaCl and (B) 4B+4_2A at 5 w/w % in 50 mM Phosphate Buffer.

FIG. 27 depicts salt concentration series for SC+6_2A at 10 w/w % (above the critical liquid crystal concentration) in pure Milli-Q water, 0.2M NaCl, 0.5M NaCl, and 1M NaCl.

FIG. 28 depicts Negative Stain TEM for SC+8_2A at 5 w/w % in 0.2M NaCl after 1 day (left) and 1 month (right).

FIG. 29 depicts salt concentration series for SC+8_2A at 5 w/w % (above the critical liquid crystal concentration) in pure Milli-Q water, 0.2M NaCl, 0.5M NaCl, and 1M NaCl.

FIG. 30 depicts Negative Stain TEM for SC+6_2A at 10 w/w % in 0.2M NaCl after 1 day (left) and 1 month (right).

FIG. 31 depicts (A) Order-order pathway study starting with SC+8_2A prepped at 5 w/w % in pure Milli-Q water, forming liquid crystals (POM, left), and after salt addition and agitation to form lattice particles (SAXS, right); and (B) Pathway study starting with SC+8_2A prepped at 10 w/w % in 0.5M NaCl, forming lattice particles (SAXS, left), and after significant solvent exchange to remove salt and agitation to induce liquid crystal formation (POM, right).

### DETAILED DESCRIPTION

All publications, patent applications, patents, and other references mentioned herein are incorporated by reference in their entirety. Unless otherwise defined, all technical and scientific terms used herein have the same meaning as commonly understood by one of ordinary skill in the art to which this invention belongs. In case of conflict, the present specification, including definitions, will control.

As used herein, the term “exemplary embodiments” refers to specific examples or embodiments of any of the aspects disclosed herein that serve as illustrations to demonstrate the various ways in which the various aspects can be implemented, constructed or practiced.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present disclosure has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

One aspect of the present disclosure is a tetrameric coiled-coil bundlemer including (i.e., comprising) four peptides, each peptide possessing a first amino acid sequence (1^{st}), a second amino acid sequence (2^{nd}), a third amino acid sequence (3^{rd}) and a fourth amino acid sequence (4^{th}), wherein the 1^{st}, 2^{nd}, 3^{rd} and 4^{th} amino acid sequence possess a heptadic peptide sequence abcdefg and are arranged as follows:

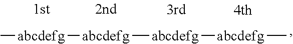

wherein (i) the 1^{st}, 2^{nd}, 3^{rd} and 4^{th} amino acid sequences together form an alpha-helical structure; (ii) the a, d, and g positions form a hydrophobic core of the bundlemer particle, wherein: the a position of the 1^{st} amino acid sequence is aspartic acid (D) and the a position of the 2^{nd}, 3^{rd} and 4^{th} amino acid sequences is alanine (A), the d position of the 1^{st}, 2^{nd}, 3^{rd} and 4^{th} amino acid sequences is isoleucine (I), and the g position of the 1^{st}, 2^{nd}, and 3^{rd} amino acid sequence is methionine (M) and the g position of the 4^{th} amino acid sequence is either methionine (M) or glutamic acid (E); (iii) each b, c, e, and f is an amino acid exposed on a surface of the bundlemer particle structure; and (iv) at least one of the b, c, e, and f positions of the 2^{nd} and/or 3^{rd} amino acid sequence is selected from a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

As used herein, the term “bundlemer” refers to a tetrameric coiled coil structure formed from four peptides possessing an alpha helical ordered structure. The bundlemers disclosed herein can be formed under physiologic conditions (e.g., in aqueous solution at pH 7.2, 22° C., under physiologically-relevant salt concentrations).

In exemplary embodiments, the bundlemer is a multi-charge (MC) bundlemer. As used herein, a “MC bundlemer” is a bundlemer that possesses multiple electric charges on its surface. A MC bundlemer can be a bundlemer that contains at least one peptide that displays both positively charged and negatively charged amino acids and/or chemical groups on its surface under physiological conditions (e.g., a peptide that displays both a lysine residue and a glutamic acid residue on its surface).

In exemplary embodiments, the bundlemer is a single-charge (SC) bundlemer. As used, a “SC bundlemer” is a bundlemer that possesses only one charge type (i.e., negative or positive) on its surface. A SC bundlemer can contain peptides that display either only positively charged amino acids and/or chemical groups on its surface or only negatively charged amino acids and/or chemical groups on its surface under physiological conditions (e.g., a bundlemer that contains peptides that (i) do not display lysine (Lys), arginine (Arg), and/or histidine (His) on their surfaces and (ii) display at least one aspartic acid (Asp) or Glutamic acid (Glu) on their surfaces; or a bundlemer that (i) contains peptides that do not display aspartic acid (Asp) or Glutamic acid (Glu) on their surfaces and (ii) display at least one lysine (Lys), arginine (Arg), and/or histidine (His) on their surfaces).

As used herein, an “amino acid” includes any compound possessing the following general structure:

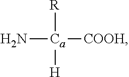

wherein the NH₂ group and/or the COOH group form an amide bond with a neighboring amino acid and wherein R includes chemical groups or side chains that are present in naturally occurring amino acids (e.g., methyl (alanine), H (glycine), or isopropyl (valine)) and chemical groups or side chains that have been modified to possess an alkene moiety, alkyne moiety or furan moiety.

The amino acids at the b, c, e, and f positions of the 1^{st}, 2^{nd}, 3^{rd} and 4^{th} amino acid sequences can be any naturally occurring amino acid such as, but not limited to, alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val).

In exemplary embodiments, at least one of the b, c, e, and f positions of the 2^{nd} and/or 3^{rd} amino acid sequence of the bundlemer is a modified lysine amino acid possessing one of the following chemical structures:

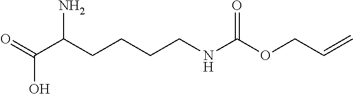 [SMILES: C=CCOC(=O)NCCCCC(N)C(=O)O]

(e.g., an amino acid possessing an alloc group or an allyloxy carbonyl moiety),

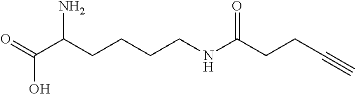 [SMILES: C#CCCC(=O)NCCCCC(N)C(=O)O]

(e.g., an amino acid possessing an alkyne moiety), or

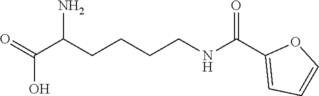 [SMILES: NC(CCCCNC(=O)c1ccco1)C(=O)O]

(e.g., an amino acid possessing a furan moiety).

In exemplary embodiments, the b position of the 1^{st} amino acid sequence is a modified lysine amino acid possessing an allyloxy carbonyl moiety.

In exemplary embodiments, each constituent peptide of the bundlemer (i.e., the 1^{st}, 2^{nd}, 3^{rd} and 4^{th} amino acid sequence of each peptide) has a charge ranging from −8 to +8. In exemplary embodiments, each tetrameric bundlemer possesses a charge ranging from −32 to +32.

In exemplary embodiments, at least one of the b, c, e, and f positions of the 2^{nd} and/or 3^{rd} amino acid sequence is a modified lysine amino acid possessing an allyloxy carbonyl moiety.

In exemplary embodiments, the bundlemer possesses a cysteine (C) before the a position of the 1^{st} amino acid sequence and, optionally, after the g position of the 4^{th} amino acid sequence.

In exemplary embodiments, the bundlemer possesses a cysteine (C) after the g position of the 4^{th} amino acid sequence.

In exemplary embodiments, the bundlemer possesses (i) a cysteine (C) before the a position of the 1^{st} amino acid sequence and after the g position of the 4^{th} amino acid sequence, and (ii) the f position of the 2^{nd} amino acid sequence and the e position of the 3^{rd} amino acid sequence are a modified lysine amino acid possessing an allyloxy carbonyl moiety.

In exemplary embodiments, the b position of the 1^{st} amino acid sequence is a modified lysine amino acid possessing an allyloxy carbonyl moiety, the f position of the 4^{th} amino acid sequence is cysteine, the e position of 3^{rd} amino acid sequence is a modified lysine amino acid possessing an allyloxy carbonyl moiety, and the 1^{st} acid sequence possess a cysteine before the a position.

In exemplary embodiments, the f position of the 2^{nd} amino acid sequence and the e position of 3^{rd} amino acid sequence are a modified lysine amino acid possessing an allyloxy carbonyl moiety.

In exemplary embodiments, the bundlemer has a diameter of about 2 nm to about 3 nm and a length of about 4 nm to about 5 nm.

As used herein, the term “about” refers to a value that is +5% of the stated value. In addition, it is understood that reference to a range of a first value to a second value includes the range of the stated values, e.g., a range of about 1 to about 5 also includes the more precise range of 1 to 5. It is also understood that the ranges disclosed herein include any selected subrange within the stated range, e.g., a subrange of about 50 to about 60 is contemplated in a disclosed range of about 1 to about 100.

In exemplary embodiments, the b position of the 2^{nd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the c position of the 2^{nd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the e position of the 2^{nd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments the f position of the 2^{nd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the b position of the 3^{rd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the c position of the 3^{rd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the e position of the 3^{rd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the f position of the 3^{rd} amino acid sequence is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the f position of the 2^{nd} amino acid sequence and the e position of 3^{rd} amino acid sequence are phenylalanine.

In exemplary embodiments, the f position of the 2^{nd} amino acid sequence and the e position of 3^{rd} amino acid sequence are a modified lysine amino acid possessing an alkyne moiety.

In exemplary embodiments, the f position of the 2^{nd} amino acid sequence and the e position of 3^{rd} amino acid sequence are tyrosine.

In exemplary embodiments, the f position of the 2^{nd} amino acid sequence and the e position of 3^{rd} amino acid sequence are tryptophan.

In exemplary embodiments, the f position of the 2^{nd} amino acid sequence and the e position of 3^{rd} amino acid sequence are a modified lysine amino acid possessing a furan moiety.

Another aspect of the present application is a second tetrameric coiled-coil bundlemer including four peptides, each peptide possessing a first amino acid sequence (1^{st}), a second amino acid sequence (2^{nd}), a third amino acid sequence (3^{rd}) and a fourth amino acid sequence (4^{th}), wherein the 1^{st}, 2^{nd}, 3^{rd} and 4^{th} amino acid sequence possess a heptadic peptide sequence abcdefg and are arranged as follows:

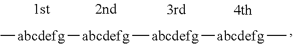

wherein (i) the 1^{st}, 2^{nd}, 3^{rd} and 4^{th} amino acid sequences together form an alpha-helical structure; (ii) the a, d, and g positions form a hydrophobic core of the tetrameric bundlemer particle structure, the a position of the 2^{nd}, 3^{rd} and 4^{th} amino acid sequences is alanine (A), the d position of the 1^{st}, 2^{nd}, 3^{rd} and 4^{th} amino acid sequences is isoleucine (I), and the g position of the 1^{st}, 2^{nd}, and 3^{rd} amino acid sequences is methionine (M); (iii) each b, c, e, and f is an amino acid exposed on a surface of the alpha-helical structure; and (iv) at least one of the b, c, e, and f positions of the 2^{nd} and/or 3^{rd} amino acid sequence is selected from the group consisting of a modified lysine amino acid possessing an allyloxy carbonyl moiety, a modified lysine amino acid possessing an alkyne moiety, a modified lysine amino acid possessing a furan moiety, phenylalanine (F), tyrosine (Y) and tryptophan (W).

In exemplary embodiments, the g position of the 4^{th} amino acid sequence is glutamic acid (E).

In exemplary embodiments, the a position of the 1^{st} amino acid sequence is alanine (A) or arginine (N).

In exemplary embodiments, the second bundlemer is a multi-charge (MC) bundlemer.

In exemplary embodiments, the second bundlemer is a single-charge (SC) bundlemer.

In exemplary embodiments, at least one of the b, c, e, and f positions of the 2^{nd} and/or 3^{rd} amino acid sequence of the second bundlemer is a modified lysine amino acid possessing one of the following chemical structures:

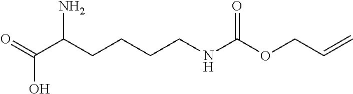 [SMILES: C=CCOC(=O)NCCCCC(N)C(=O)O]

(e.g., an amino acid possessing an alloc group or an allyloxy carbonyl moiety),

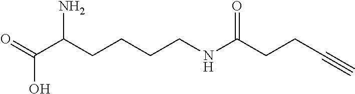 [SMILES: C#CCCC(=O)NCCCCC(N)C(=O)O]

(e.g., an amino acid possessing an alkyne moiety), or

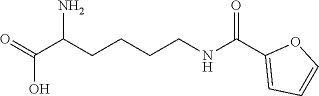 [SMILES: NC(CCCCNC(=O)c1ccco1)C(=O)O]

(e.g., an amino acid possessing a furan moiety).

In exemplary embodiments, the b position of the 1^{st} amino acid sequence of the second bundlemer is a modified lysine amino acid possessing an allyloxy carbonyl moiety.

In exemplary embodiments, each constituent peptide of the second bundlemer (i.e., the 1^{st}, 2^{nd}, 3^{rd} and 4^{th} amino acid sequence of each peptide) has a charge ranging from −8 to +8. In exemplary embodiments, each tetrameric bundlemer possesses a charge ranging from −32 to +32.

In exemplary embodiments, at least one of the b, c, e, and f positions of the 2^{nd} and/or 3^{rd} amino acid sequence of the second bundlemer is a modified lysine amino acid possessing an allyloxy carbonyl moiety.

In exemplary embodiments, the second bundlemer possesses a cysteine (C) before the a position of the 1^{st} amino acid sequence and, optionally, after the g position of the 4^{th} amino acid sequence.

In exemplary embodiments, the second bundlemer possesses a cysteine (C) after the g position of the 4^{th} amino acid sequence.

In exemplary embodiments, the second bundlemer possesses (i) a cysteine (C) before the a position of the 1^{st} amino acid sequence and after the g position of the 4^{th} amino acid sequence, and (ii) the f position of the 2^{nd} amino acid sequence and the e position of the 3^{rd} amino acid sequence are a modified lysine amino acid possessing an allyloxy carbonyl moiety.

In exemplary embodiments, the b position of the 1^{st} amino acid sequence of the second bundlemer is a modified lysine amino acid possessing an allyloxy carbonyl moiety, the f position of the 4^{th} amino acid sequence is cysteine, the e position of 3^{rd} amino acid sequence is a modified lysine amino acid possessing an allyloxy carbonyl moiety, and the 1^{st} acid sequence possess a cysteine before the a position.

In exemplary embodiments, the f position of the 2^{nd} amino acid sequence and the e position of 3^{rd} amino acid sequence of the second bundlemer are a modified lysine amino acid possessing an allyloxy carbonyl moiety.

In exemplary embodiments, the second bundlemer has a diameter of about 2 nm to about 3 nm and a length of about 4 nm to about 5 nm.

In exemplary embodiments, the b position of the 2^{nd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the c position of the 2^{nd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the e position of the 2^{nd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments the f position of the 2^{nd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the b position of the 3^{rd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the c position of the 3^{rd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the e position of the 3^{rd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the f position of the 3^{rd} amino acid sequence of the second bundlemer is either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W).

In exemplary embodiments, the f position of the 2^{nd} amino acid sequence of the second bundlemer and the e position of 3^{rd} amino acid sequence are phenylalanine.

In exemplary embodiments, the f position of the 2^{nd} amino acid sequence of the second bundlemer and the e position of 3^{rd} amino acid sequence are a modified lysine amino acid possessing an alkyne moiety.

In exemplary embodiments, the f position of the 2^{nd} amino acid sequence and the e position of 3^{rd} amino acid sequence of the second bundlemer are tyrosine.

In exemplary embodiments, the f position of the 2^{nd} amino acid sequence and the e position of 3^{rd} amino acid sequence of the second bundlemer are tryptophan.

In exemplary embodiments, the f position of the 2^{nd} amino acid sequence and the e position of 3^{rd} amino acid sequence of the second bundlemer are a modified lysine amino acid possessing a furan moiety.

In exemplary embodiments, the four peptides of the bundlemer possess one of the following amino acid sequences:

| | | 1st amino | 2nd amino | 3rd amino | 4th amino | |
|---|---|---|---|---|---|---|
| Sequence | | acid | acid | acid | acid | |
| No. | X | sequence | sequence | sequence | sequence | Y |
| SEQ. ID. | C | DKEIRRM | AEKIRK¹M | AERIKQM | AEQIYKE | A |
| NO. 1 | | | | | | |
| SEQ. ID. | C | DKEIRRM | AEKIRK¹M | AERIK¹QM | AEQIYKE | A |
| NO. 2 | | | | | | |
| SEQ. ID. | — | DK¹EIRRM | AEKIRKM | AERIK¹QM | AEQIYKE | A |
| NO. 3 | | | | | | |
| SEQ. ID. | C | DKEIRRM | AEKIRK¹M | AERIK¹QM | AEQIYCE | AC |
| NO. 4 | | | | | | |
| SEQ. ID. | C | DK¹EIRRM | AEKIRKM | AERIK¹QM | AEQIYCE | A |
| NO. 5 | | | | | | |
| SEQ. ID. | — | DKEIRRM | AEEIRK¹M | AERIK¹QM | AEQIYKE | A |
| NO. 6 | | | | | | |
| SEQ. ID. | — | DKEIRRM | AEEIRK²M | AERIK²QM | AEQIYKE | A |
| NO. 7 | | | | | | |
| SEQ. ID. | — | DKEIRRM | AEEIRK³M | AERIK³QM | AEQIYKE | A |
| NO. 8 | | | | | | |
| SEQ. ID. | — | DKEIRRM | AEEIRFM | AERIFQM | AEQIYKE | A |
| NO. 9 | | | | | | |
| SEQ. ID. | — | DKEIRRM | AEEIRWM | AERIWQM | AEQIYKE | A |
| NO. 10 | | | | | | |
| SEQ. ID. | — | DKEIRRM | AEEIRYM | AERIYQM | AEQIQKE | A |
| NO. 11 | | | | | | |
| SEQ. ID. | — | NTTIQKM | ATNIRK¹M | ATSIK¹KM | ATTIYKQ | A |
| NO. 12 | | | | | | |
| SEQ. ID. | — | ARTIQTM | ASKIRK¹M | ATSIK¹KM | ATTIYKQ | A |
| NO. 13 | | | | | | |

wherein X is/are amino acid residues present before the 1^{st} amino acid sequence, Y is/are amino acid residues present after the 4^{th} amino acid sequence, K¹ is a modified lysine amino acid possessing an allyloxy carbonyl moiety, K² is a modified lysine amino acid possessing an alkyne moiety, and K³ is a modified lysine amino acid possessing a furan moiety.

Another aspect of the present disclosure is an amorphous network including one or more bundlemers disclosed herein that (i) have a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W) at at least one of the b, c, e, or f positions of the 2^{nd} and/or 3^{rd} amino acid sequence; and (ii) have at least one cysteine residue either before the 1^{st} amino acid sequence and/or after the g position of the 4^{th} amino acid sequence.

In exemplary embodiments, the amorphous network possesses a storage modulus (G′) of about 50 kPa to about 2,300 kPa. In exemplary embodiments, the amorphous network possesses a storage modulus above 2,300 kPa. The storage modulus of the amorphous network is dependent upon the number and the positions of the crosslinkable groups on the bundlemers. With this knowledge, those of ordinary skill in the art can readily appreciate that storage moduli above 2,300 kPa can be achieved with embodiments wherein the bundlemer contains a high number of crosslinkable groups (e.g., cysteines and lysine amino acids possessing an allyloxy carbonyl moiety, which can participate in thiol-ene crosslinking reactions).

Another aspect of the present disclosure is a lattice nanostructure including one or more bundlemers disclosed herein that have a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W) at at least one of the b, c, e, or f positions of the 2^{nd} and/or 3^{rd} amino acid sequence; and do not possess a cysteine residue before the 1^{st} amino acid sequence and after the g position of the 4^{th} amino acid sequence.

The inventors have surprisingly discovered that bundlemers possessing either a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W) at at least one of the b, c, e, or f positions of the 2^{nd} and/or 3^{rd} amino acid sequence were capable of forming lattice nanostructures, which were not observed in embodiments possessing cysteine residue before the 1^{st} amino acid sequence and after the g position of the 4^{th} amino acid sequence.

In exemplary embodiments, the lattice nanostructure includes more than one bundlemer that possesses a modified lysine amino acid possessing an allyloxy carbonyl moiety at at least one of the b, c, e, or f positions of the 2^{nd} and/or 3^{rd} amino acid sequence, and possesses an average pore size of about 5 to about 6 nm.

In exemplary embodiments, the lattice nanostructure includes more than one bundlemer that possesses a modified lysine amino acid possessing an allyloxy carbonyl moiety at at least one of the b, c, e, or f positions of the 2^{nd} and/or 3^{rd} amino acid sequence, and possesses a truss-like face-centered cubic (FFC) lattice symmetry.

In exemplary embodiments, the lattice nanostructure includes more than one bundlemer that possesses a modified lysine amino acid possessing an allyloxy carbonyl moiety at at least one of the b, c, e, or f positions of the 2^{nd} and/or 3^{rd} amino acid sequence, and is found within diamond shaped particle morphology.

In exemplary embodiments, the lattice nanostructure includes more than one bundlemer that possesses a modified lysine amino acid possessing an allyloxy carbonyl moiety at at least one of the b, c, e, or f positions of the 2^{nd} and/or 3^{rd} amino acid sequence, and is found within rectangular shaped particle morphology.

In exemplary embodiments, the lattice nanostructure includes more than one bundlemer that possesses a modified lysine amino acid possessing an alkyne moiety, phenylalanine or tyrosine at at least one of the b, c, e, or f positions of the 2^{nd} and/or 3^{rd} amino acid sequence, and possesses an average pore size of about 2 to about 3 nm.

In exemplary embodiments, the lattice nanostructure includes more than one bundlemer that possesses a modified lysine amino acid possessing an alkyne moiety, phenylalanine or tyrosine at at least one of the b, c, e, or f positions of the 2^{nd} and/or 3^{rd} amino acid sequence, and possesses chains of end-to-end stacked bundlemer particles aligned on a square lattice.

Another aspect of the present disclosure is a rounded nanostructure that includes more than one bundlemer that possesses a tryptophan at at least one of the b, c, e, or f positions of the 2^{nd} and/or 3^{rd} amino acid sequence.

In exemplary embodiments, the rounded nanostructure possesses an outer diameter of about 10 to about 13 nm, an inner diameter of about 5 to about 6 nm and/or an average thickness of about 2 nm.

Another aspect of the present disclosure is a polymorphic lattice that includes more than one bundlemer that possesses a modified lysine amino acid possessing a furan moiety at at least one of the b, c, e, or f positions of the 2^{nd} and/or 3^{rd} amino acid sequence.

Another aspect of the present disclosure is a liquid crystalline structure that contains more than one SC bundlemer that possesses a modified lysine amino acid possessing an allyloxy carbonyl moiety at at least one of the b, c, e, or f positions of the 2^{nd} and/or 3^{rd} amino acid sequence.

In exemplary embodiments, the liquid crystalline structure possesses a critical concentration for liquid crystal formation at or below 5 w/w %.

Another aspect of the present application is a method of creating an amorphous network that includes one or more of: (i) solubilizing more than one bundlemer that (i) have a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W) at at least one of the b, c, e, or f positions of the 2^{nd} and/or 3^{rd} amino acid sequence; and (ii) have at least one cysteine residue either before the 1^{st} amino acid sequence and/or after the g position of the 4^{th} amino acid sequence in an aqueous solution; (ii) adding at least one photoinitiator into the aqueous solution; and (iii) irradiating the solution to promote thiol-ene click chemistry reactions between the allyloxy carbonyl moieties and cysteines of the bundlemers.

The at least one photoinitiator can be any radical generating photoinitiator commonly used and/or known in the art. The photoinitiator can be selected from, but is not limited to, alpha-hydroxy ketones (e.g., Irgacure 2959), phosphine oxides (e.g., Lithium phenyl-2,4,6-trimethylbenzoylphosphinate (LAP)), thioxanthones, quaternary ammonium salts (e.g., VA-086), dye-sensitized systems (e.g., riboflavin, Eosin Y), and onium salts (e.g., diaryliodonium and triarylsulfonium salts)

In exemplary embodiments, the solution is irradiated with the appropriate wavelength of light (such as, but not limited to, UV light and visible light).

The aqueous solution can be pure water (i.e., just water with no other substances dissolved in the solution) or can be a buffer solution (i.e., a solution that contains one or more salts to help regulate the pH of the solution and/or assist with protein folding). In exemplary embodiments, the aqueous solution contains one or more phosphates, tris(hydroxymethyl)aminomethane, acetates, formates, trifluoroacetic acid, or 4-(2-hydroxyethyl)-1-piperazineethanesulfonic acid, 3-(N-Morpholino) propanesulfonic acid.

In exemplary embodiments, the aqueous solution contains about 0.25 g/mL to about 0.75 g/mL of the bundlemers.

In exemplary embodiments, the aqueous solution contains a bundlemer concentration ranging from about 25 wt % to about 75 wt % before the irradiating step.

In exemplary embodiments, the aqueous solution contains about 0.5 mM to about 40 mM of the UV light photoinitiator after (ii).

Another aspect of the present disclosure is a method of creating a nanostructure that includes one or more of: (i) solubilizing more than one bundlemer that have a modified lysine amino acid possessing an allyloxy carbonyl moiety, a modified lysine amino acid possessing an alkyne moiety, a modified lysine amino acid possessing a furan moiety, phenylalanine (F), tyrosine (Y) or tryptophan (W) at at least one of the b, c, e, or f positions of the 2^{nd} and/or 3^{rd} amino acid sequence; and do not possess a cysteine residue before the 1^{st} amino acid sequence and after the g position of the 4^{th} amino acid sequence into an aqueous solution; adding at least one photoinitiator into the aqueous solution; and irradiating the solution to promote crosslinking reactions.

Another aspect of the present disclosure is a method for forming LC structures that includes solubilizing more than one bundlemer that have a modified lysine amino acid possessing an allyloxy carbonyl moiety at at least one of the b, c, e, or f positions of the 2^{nd} and/or 3^{rd} amino acid sequence; and do not possess a cysteine residue before the 1^{st} amino acid sequence and after the g position of the 4^{th} amino acid sequence into an aqueous solution, wherein the aqueous solution contains a salt concentration below 0.5 M.

Another aspect of the present application is an article formed from any one of the structures disclosed herein.

In exemplary embodiments, the article is a macromolecular material selected from catalytic membranes, chiral compound separators, ion separators, barrier plastics, nanostructured films for membranes, fuel cells, batteries, chemical separators, water desalination devices, biomedical devices, drug delivery devices, smart coatings in implants and diagnostic equipment, or cell scaffolds for tissue engineering.

### EXAMPLES

The present disclosure will be described in more detail with reference to the following Examples, which shows exemplary embodiments in accordance with the present disclosure. The present disclosure is not limited to these exemplary embodiments.

### Example 1: Peptide Bundlemer Networks Driven by Alloc Side Chains

### Materials and Methods Used in Example 1

### Materials

Amino acids were purchased from CEM. MilliQ water was acquired via treatment of reverse osmosis water with a MilliPore DIRECT-Q3 system. Lithium acylphosphinate (LAP) and phosphate buffer (pH 7) were purchased from Thermo Scientific.

### Solid Phase Peptide Synthesis (SPPS)

Peptides were synthesized via Fmoc solid phase peptide synthesis (SPPS) using a CEM Liberty Blue. Fmoc protected amino acids were purchased from CEM. Amino acids were prepared at 0.2 M and rink amide resin was used. Peptides were cleaved in a cocktail of 95% trifluoroacetic acid (TFA), 2.5% triisopropylsilane (TIPS), and 2.5% MilliQ. For peptides containing cysteine, 5% w/v of dithiothreitol (DTT) was added. Cleavage was conducted for 2-3 hours while shaking. After cleavage, solution was precipitated dropwise into chilled diethyl ether and centrifuged. Two additional washes with diethyl ether were conducted. Peptide purification was conducted using a Waters LC Prep 150 Reverse-phase High Performance Liquid Chromatography (HPLC) system with a mobile phase of 0.1% trifluoroacetic acid in water and 0.1% trifluoroacetic acid in acetonitrile. Peptides were purified using a semi-prep C-18 column with flow rates of 30 mL/min. A gradient of 1%/min and a range of 80% to 40% water was used. Pure fractions were lyophilized and stored at −20° C. Ultra performance liquid chromatography-electrospray ionization-tandem mass spectrometry (UPLC-ESI-MS/MS) was used to confirm the molecular weight of products via Xevo LC-MS ESI Mass Spectrometer.

### Circular Dichroism (CD) Spectroscopy

CD spectroscopy measurements were conducted using a JASCO 1500 CD spectrometer. Solutions were prepped at 0.1 mM. Measurements were collected from 250 nm to 190 nm wavelength and 3 accumulations were averaged. A quartz cuvette with a 1 mm pathlength was used. Data was collected at 20° C.

### Thiol-Ene Reaction

Reactions were conducted in MilliQ water with a photo initiator concentration of 20 mM lithium acylphosphinate (LAP). Solutions were prepared at small volumes (less than 100 μL), and reported concentrations are in weight of solids/volume of liquid. Peptide concentrations studied included 0.25 g/mL, 0.50 g/mL, or 0.75 g/mL. Solutions were prepped by (1) centrifuging lyophilized peptide to create a pellet, (2) adding appropriate volume of photoinitator solution (water), and (3) conducting a series of vortex mixing, centrifugation, and manual mixing to make a homogeneous concentrated solution. Sequences reported have high solubility in water, even at these high concentrations. An Omnicure Series S2000 UV high pressure mercury lamp equipped with a liquid light guide was used to irradiate the specimens. The wavelength of 365 nm was selected using a band pass interference filter and intensity of 15 mW/cm² was measured using a radiometer. Exposure time varied for experiments.

### Rheometry

Oscillatory rheology measurements were conducted on a HR30 rheometer using a 2% strain amplitude and 2 rad/s frequency. Parallel plate geometry was used with an 8 mm diameter sandblasted geometry. An Omnicure Series S2000 was used for irradiation at 15 mW/cm² and 365 nm wavelength. A geometry gap of 150-200 μm was used. Three experiments were run at each concentration and average moduli and standard error is reported. Statistical analysis was conducted using the G′ and G″ at the time point of maximum G′. The rheometry plot with the highest modulus for each sequence is plotted in the figures in the main text for comparison.

### Transmission Fourier Transform Infrared Spectroscopy (FTIR)

FTIR measurements were conducted on a Thermo Nicolet Nexus 470 FTIR Spectrometer in transmission mode. A calcium fluoride holder with a gap of 10 μm was used to conduct transmission experiments for an aqueous solution. A range of 1200 cm 1 to 2800 cm 1 was scanned and a mercury calcium telluride (MCT-A) detector was used. Chamber was purged under nitrogen for at least 15 minutes for each sample. Resolution was 1 and 128 scans were conducted for each sample.

### Transmission Electron Microscopy (TEM)

TEM images were obtained on a FEI TALOS F200C microscope using 200 kV accelerating voltage. 200 mesh carbon coated copper grids (Electron Microscopy Solutions Inc.) were glow discharge treated using a PELCO easiGlow™ 91000 Glow Discharge Cleaning System. Samples were prepped by depositing 5 μL of peptide solution (diluted to ˜0.3-1 wt/vol %) concentration) on glow discharge treated grids. Deposition time was 1 minute before removing solution. 5 μL of 2 wt % phosphotungstic acid stain was then added to the grid for 50 seconds and removed with a KimWipe. Grids were air dried for at least 10 minutes prior to being imaged.

For cryo-TEM, the same microscope was used. Type C Lacey carbon grids (Ted Pella, Inc.) were glow discharge treated for 30 seconds. A Vitrobot vitrification system was used for sample prep of 5 μL of solution with varied blotting conditions for optimal sample thickness in a 100% humidity chamber. Samples were frozen in liquified ethane and transferred (quickly) to a liquid nitrogen bath.

### Small Angle X-Ray Scattering (SAXS)

SAXS experiments were conducted using 2 different sources. Both sources demonstrated the same scattering behavior of solutions, therefore the higher energy data was reported.

SAXS experiments were conducted on DND-CAT beamline at Argonne National Laboratory using the Advanced Photon Source. Peptide solutions were prepped at 5 wt/vol %, heated to 90° C. for several hours, cooled and transferred to quart capillaries with 1 mm diameter. Beam energy used is 10 keV (wavelength is 1.2398 Angstroms). The exposure time varied for samples.

### Coarse-Grained Model and Bayesian Optimization

Bayesian optimization (BO) is a sequential design strategy for global optimization of “black-box” functions where explicit analytical expressions and gradient information may be difficult or impractical (e.g., see Snoek, J.; Larochelle, H.; Adams, R. P. Practical Bayesian Optimization of Machine Learning Algorithms. Adv. Neural Inf. Processing Syst. 2012, 25; and Nogueira, F. Bayesian Optimization: Open Source Constrained Global Optimization Tool for Python. 2014). The method is particularly well-suited for optimizing expensive-to-evaluate functions and navigating high-dimensional search spaces. This makes it useful for refine the parameters defined in crystal structure analysis methods, such as Rietveld refinement (e.g., see Rietveld, Hugo M. A Profile Refinement Method for Nuclear and Magnetic Structures. J. Crystallogr. 1969, 2, 65-71).

In this example, similar ideas from Rietveld refinement were adopted to interpret the structure of protein assemblies by fitting small angle X-ray scattering (SAXS) diffraction data with structure factors calculated from a coarse-grain (CG) model. An optimization was performed to search for the best simulated assembly parameters that agree with the experimental results. The CG model contained the CG bundlemers and refinement parameters specifying particular lattice structures, including the positions of CG bundlemers in a box with periodic boundaries. Each CG bundlemer is constructed by evenly placing 16 beads on the surface of a cylinder, which has length of approximately 4.5 nm and radius of approximately 2 nm to match with the physical bundlemer. Once the CG model lattice was constructed, the following equation was used to estimate the structure factor.

$$S ( q ) = S ( q ) = \frac{1}{N} {\sum }_{j = 1}^{N} {\sum }_{k = 1}^{N} {e}^{- iq \cdot ( {R}_{j} - {R}_{k} )}$$

Isotropic orientation of sublattices was assumed, and the structure factor depended only on the magnitude of q=/q/. The sum in each case was a sum over scattering centers, and Ri is the position of the scattering center i. N is the number of scattering centers. Cases where (a) Ri is the center of mass position for each bundlemer and the sum is over bundlemers, and (b) Riis the position of each bead and the sum is over all beads in all bundlemers were examined. The optimization was employed to determine the positions of the bundlemers in the CG model that best recover experimental structure factor, as quantified by a mean absolute error

$$( MAE ) , MAE = \frac{1}{5} \sum \vert \frac{2 π}{{q}_{saxs}} - \frac{2 π}{{q}_{cg}} \vert ,$$

where qₛₐₓₛ and q_{cg} correspond to the locations of each of the first 5 peaks in the corresponding S (q), as determined by experimental SAXS diffraction data and the coarse-grain model, respectively.

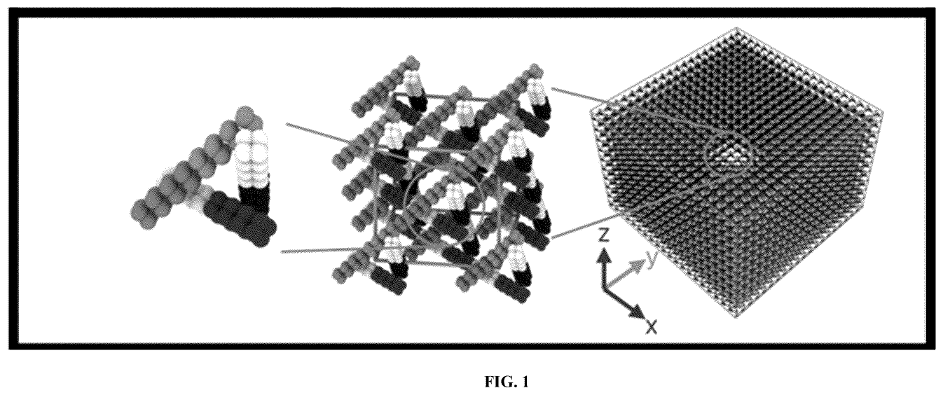

Initially, a candidate lattice was specified by providing a starting set of parameters for the CG model. Previous knowledge from the SAXS diffraction data indicated that the lattice packing exhibits FCC characteristics. With TEM as a guide, the assembled structure was posited to closely resemble the FCC truss lattice, prompting the modeling of the relative orientations of the CG bundlemers similarly. A small portion of lattice consisting of 6 CG bundlemers was created and grouped into three pairs. Within each pair, the two CG bundlemers colinearly align end-to-end throughout the optimization process. The refinement parameter, L, is the distance between the center of mass (COM) of the two CG bundlemers in each pair. Next, one pair along the y axis was fixed and the other two pairs were generated so as to match the relative strut orientations within the FCC truss lattice (see FIG. 1). The variable lattice parameters are L and the Cartesian coordinates of each of the three bundle pairs in the lattice repeat unit (hexamer).

These choices specified a set of refinement parameters {xᵢ, y, zᵢ, L} where “i” labels each bundlemer pair. This replicated and translated to generate a subset of the lattice. Once an initial lattice piece was structured, the lattice piece was replicated four times along each Cartesian dimension, using the refinement parameter L and predetermined rotational directions. The size of the lattice was selected to minimize finite size effects for the q-values considered; systems with larger box sizes gave similar results. In total, 10 refinement paraments were produced. After establishing the setup, the search space was defined for each parameter: L ranged from 4.4 to 5 nm, while xᵢ, y, and zᵢ each ranged from −5 to 5 nm.

In the optimization process, a lattice comprising 100 CG models was initially generated and the optimization was ran for 300 steps, which was sufficient to obtain a mean absolute error less than 0.06 nm. Bayesian packages with the upper confidence bound (UCB) as an acquisition function and Gaussian Process Regressor (GPR) as the surrogate model were used (e.g., see Snoek, J.; Larochelle, H.; Adams, R. P. Practical Bayesian Optimization of Machine Learning Algorithms. Adv. Neural Inf. Processing Syst. 2012, 25; and Nogueira, F. Bayesian Optimization: Open Source Constrained Global Optimization Tool for Python. 2014). The parameter values with lowest MAE were (in units of nm): L=4.80, x₁=2.02, y₁=1.16, z₁=1.82, x₂=−2.45, y₂=3.78, z₂=1.96, x₃=−0.16, y₃=4.79, z₃=0.98.

The choice of scattering centers used to calculate S (q), calculated as a sum over bundles or as a sum over beads, did not affect the lattice parameters identified upon optimization nor the q values of the first five peaks in S (q).

### CG Simulation for Bond Formation

To simulate the bond formation process in bundlemer networks, coarse-grained (CG) simulations were used. In these simulations, bundlemers were represented as rigid cylinders with a height of 8σ and a diameter of 4σ, where σ is the diameter of beads within the bundlemer. Each bundlemer core was treated as a rigid body and possessed no internal degrees of freedom. Reactive sites were introduced on the bundlemer's surface according to the positions of cysteine and alloc groups. In the CG model, four bead types depict the bundlemer, allyloxycarbonyl (alloc) protected lysine, cysteine, and water. The bonds to reactive beads obeyed a harmonic potential, and no angle potential terms were present. For the CG bundlemer of 2A2C, cysteine beads were treated as rigid in tandem with the bundlemer. Eight alloc-protected lysine beads formed a ring around the bundle center. For 2A2CM, four cysteine beads were unconstrained, with two located at each end, as indicated by the bonds. Nonbonded interaction between all beads were specified by a purely repulsive Weeks-Chandler-Andersen (WCA) truncated Lennard-Jones potential with energy and length parameters ∈, σ. Harmonic bonds were represented by the well-known potential, U_{bond}(r)=k(r−r₀)², with k=1000∈/σ² and r₀=1σ.

The simulation was initiated by randomly placing CG bundlemers (400 bundlemers in total) into a large simulation box. Water beads were added such that the number of water beads and bundlemer beads were equal, approximating 50 wt % bundlemers and 50 wt % water. Initially, the system density was low. The LAMMPS MD simulation package was employed (e.g., see Thompson, A. P. et al. LAMMPS-a Flexible Simulation Tool for Particle-Based Materials Modeling at the Atomic, Meso, and Continuum Scales. Comput. Phys. Commun. 2022, 271), utilizing the velocity-Verlet algorithm and a Langevin thermostat within an NVT ensemble at a reduced (unitless) temperature of T=1.0, running a preliminary simulation for 30,000τ. Temperature and time are reported in unitless (reduced) units:

$$T \to kT / ϵ and τ \to t \sqrt{\frac{ϵ}{m {σ}^{2}}} ,$$

where ∈, σ are the parameters associated with the purely repulsive (WCA) Lennard-Jones interaction between beads and m is the mass of one bead. Subsequently, an NPT ensemble was used for equilibration over another 30,000τ. Then, a production run for 150,000 τ was conducted using the bond creation algorithm provided by LAMMPS along with an NPT ensemble to mimic the experimental thiol-ene bond formation process. Within this bond creation phase, bonding was allowed between cysteine beads and alloc-protected lysine beads. At any time step, an irreversible bond formed with probability 0.5 if the distance between complementary reactive beads was less than 1.1 σ. As can be seen in the results, inter-bundlemer bonds were much more likely for 2A2C than for 2A2CM.

### All-Atom Simulations

To better understand the local interaction between bundlemers mediated by the alloc groups, all-atom simulations were performed for the bundlemers. In the monomer simulation, the bundlemer was centered in water box whose dimensions were selected to provide a minimum distance 15 Å from the box face to the nearest peptide atom. After constructing the initial configuration containing the bundlemer, counter ions to achieve charge neutrality, and TIP-3P water, 100000 steps of steepest descent energy minimization were performed, then the systems simulated for 10 ns with an NVT ensemble at temperatures T=300K. Subsequently, the production run in the NPT ensemble was executed for 300 ns using the integration timestep of 0.002 ps at selected temperature T=300K and pressure of P=1 bar. During the NPT production, a velocity-rescaling algorithm (see Bussi, G.; Donadio, D.; Parrinello, M. Canonical Sampling through Velocity Rescaling. J. Chem. Phys. 2007, 126) with a time constant of 0.1 ps, and Parrinello-Rahman barostat (see Parrinello, M.; Rahman, A. Crystal Structure and Pair Potentials: A Molecular-Dynamics Study. Phys. Rev. Lett. 1980, 45, 1196-1199) with a coupling constant of 2.0 ps, were applied to maintain constant temperature and pressure, respectively. The Verlet algorithm was used to update the positions of atoms. Gromacs and Charmm36 force field (see Abraham, M. J.; Murtola, T.; Schulz, R., Páll, S.; Smith, J. C.; Hess, B.; & Lindahl, E. GROMACS: High Performance Molecular Simulations through Multi-Level Parallelism from Laptops to Supercomputers. SoftwareX 2015, 1-2, 19-25; and Huang, J.; Mackerell, A. D., Jr. CHARMM36 All-Atom Additive Protein Force Field: Validation Based on Comparison to NMR Data. J. Comput. Chem. 2013, 34, 2135-2145) were used conduct all the simulations. The alloc group was parametrized using CHARMM General Force Field (see Vanommeslaeghe, K.; Hatcher, E.; Acharya, C.; Kundu, S.; Zhong, S.; Shim, J.; Darian, E.; Guvench, O.; Lopes, P.; Vorobyov, I.; Mackerell, A. D., Jr. CHARMM General Force Field: A Force Field for Drug-Like Molecules Compatible with the CHARMM All-Atom Additive Biological Force Fields. J. Comput. Chem. 2010, 31, 671-690). Pymol and Ovito were used for visualization purposes (see Lilkova, E.; Petkov, P.; Ilieva, N.; Litov, L. The PyMOL Molecular Graphics System, Version 2.0. Schrödinger, LLC. 2015; and Stukowski, A. Visualization and Analysis of Atomistic Simulation Data with OVITO—the Open Visualization Tool. Model. Simul. Mater. Sci. Eng. 2009, 18, 015012).

From the simulations, the bundlemer retained the original tetrahelical structure. Overall, the dominant presentation of the alloc groups has them separated and extending outward from the center of the bundle.

In the dimer simulations, given the uncertainty regarding the precise contact among alloc groups during assembly, three distinct initial configurations were generated. In these configurations, the dimers are orthogonal to each other. Each initial configuration represents different contact sides of alloc groups achieved by rotating the bundlemers along the anisotropic axis. After constructing the initial configuration, the same simulation procedures as those used for monomer simulations were applied. After 300 ns, that one of the configurations maintained stable contact throughout the entire simulation. The remaining replicas, however, did not exhibit stability and eventually separated after 300 ns.

### EXPERIMENTS AND RESULTS

### Experiment 1: Controllable Covalent Crosslinked Peptide Networks

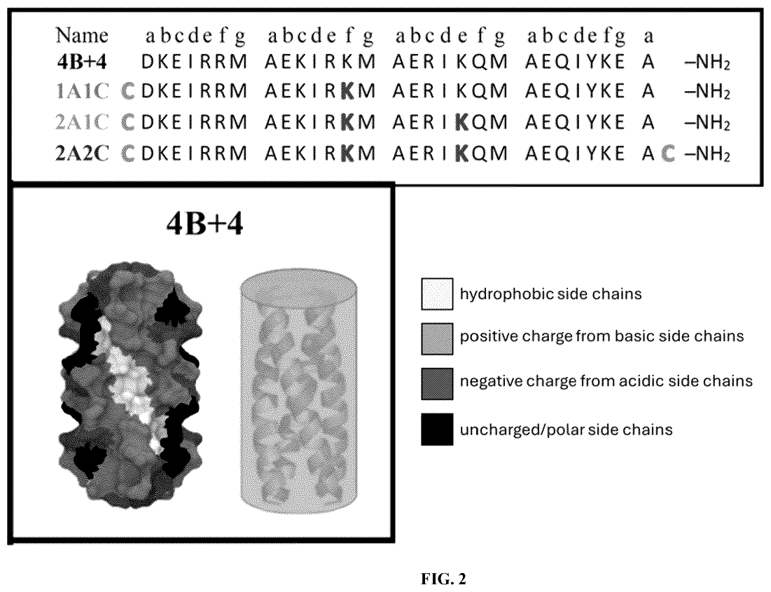

To directly observe the impact of the number of possible crosslinking sites on network formation, a single bundlemer sequence, designated 4B+4 (SEQ. ID. NO. 14), was chosen for modification with different numbers of alloc side chains and cysteines for photoinitiated thiol-ene crosslinking (see FIG. 2). This sequence was originally designed for its overall net electrostatic charge of +4 per peptide or +16 per homotetrameric bundlemer at neutral pH. Locations for the alloc-protected lysines were chosen based on existing lysine locations in the original peptide design to prevent the destabilization of the coiled-coil structure. Cysteines were added to the constituent peptide termini regions to ensure the thiols were exposed and accessible for crosslinking and to eliminate the potential for intrabundle thiol-ene reactions. All sequences are confirmed to form stable alpha helical coiled-coil bundles at 20° C. using circular dichroism (CD) spectroscopy with minima at 208 nm and 222 nm. Although these sequences have a variety of side chain functionality and reactive specificity, they all form the same bundlemer building block nanoparticle, approximately 2 nm in diameter by 4 nm in length. This design capability, based on the stability of the bundlemer to amino acid substitutions, is critical to modulate the reactive group spatial display from the exterior of the building blocks.

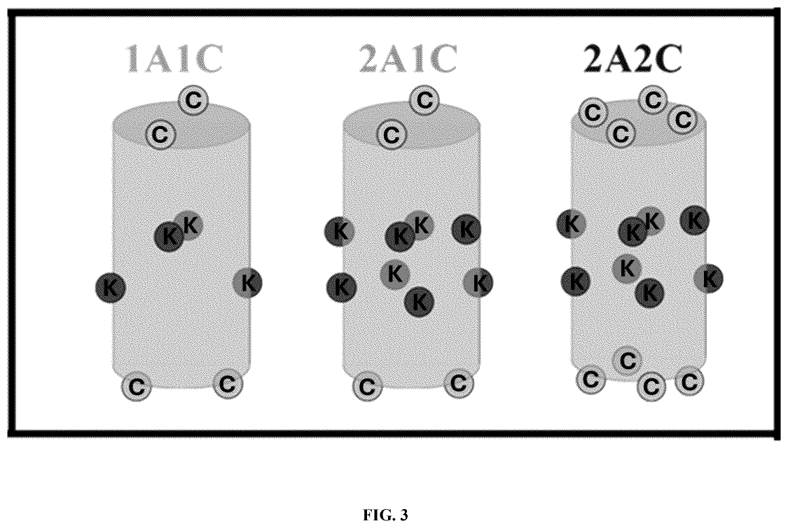

Solutions were prepared with the addition of a water-soluble UV light photoinitiator and FTIR measurements were conducted to confirm the alpha helical structure of the peptides before and after UV crosslinking. The alpha helical structure, represented by a peak at 1653-1654 cm-1, is confirmed to be conserved before and after UV irradiation. The locations of the different reactive sites are shown on the cylindrical schematic of each folded bundlemer in FIG. 3.

Sequences 1A1C, 2A1C, and 2A2C (see FIG. 2) were chosen to study how the number of reactive sites and ratio of reactive groups affects the mechanical properties of the resulting crosslinked networks. 1A1C contains one alloc group towards the middle of each constituent bundlemer peptide and one cysteine at the peptide N-terminus. Since the bundlemers are homotetrameric, there is the possibility of 8 interparticle covalent interactions. 2A1C adds an additional alloc per peptide for an asymmetric number of alloc groups and thiol functional groups. Finally, 2A2C adds an additional cysteine as the C-terminal amino acid, and thus provides the possibility of up to 16 covalent interactions between neighboring reactive particles.

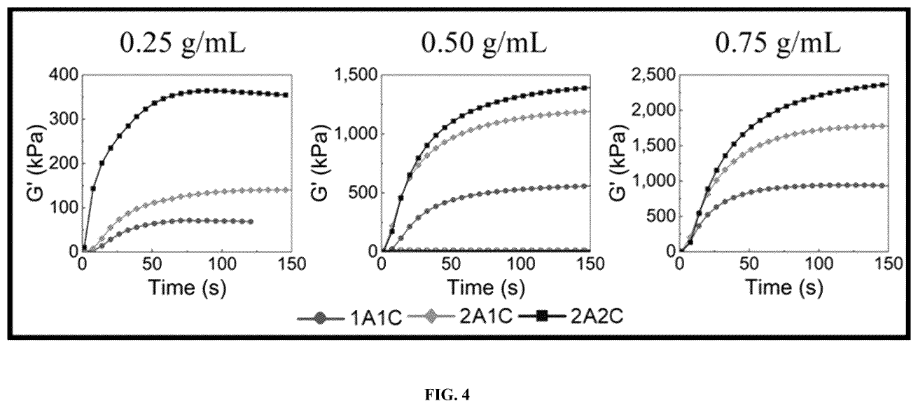

A range of concentrations were examined with rheological analysis for each sequence to observe the effect of bundlemer concentration on film crosslinking. Experiments were conducted at 0.25 g/mL, 0.5 g/mL, and 0.75 g/mL of the respective peptide bundlemer particles in aqueous solution with a photoinitiator concentration of 20 mM. All experiments were run in triplicates with the highest modulus run at all concentrations reported in FIG. 4. As expected, rheometry (see Table 1) reveals a clear modulus increase with increasing peptide concentration for all sequences, indicative of the increase in interbundlemer crosslinks with increasing building block concentration.

TABLE 1
Storage Modulus (kPa) and Standard Error (kPa)
Calculated from Triplicates of Each Sequence
at 0.25 g/mL, 0.50 g/mL, and 0.75 g/mL

Storage Modulus (G′, kPa)

| Concentration | 1A1C | 2A1C | 2A2C |
|---|---|---|---|
| 0.25 g/mL | 68 ± 2 | 130 ± 10 | 340 ± 10 |
| 0.50 g/mL | 510 ± 40 | 1,250 ± 30 | 1,300 ± 60 |
| 0.75 g/mL | 840 ± 50 | 1,750 ± 20 | 2,300 ± 80 |

With shear storage moduli approaching 2.5 MPa for the highest concentrated 2A2C network (0.75 g/mL), the bundlemer building blocks formed stiff, hydrated networks when crosslinked at high concentrations. For all sequences at all concentrations, the storage modulus (G′) was observed to be much larger than the loss modulus (G″). These results are similar with mechanical properties observed in other, concentrated, physical protein networks including elastin-like polypeptides (ELPs) networks (G′=0.1-1.0 MPa, 20 wt %) and beta-sheet tandem-repeat proteins inspired by squid (G′=0.1-10 MPa, 80 wt %). Importantly, the three sequences consistently exhibit the following rank in network shear modulus at each respective bundlemer concentration, 1A1C <2A1C <2A2C. This relative modulus increase is consistent with the number of possible interbundle crosslinks, assuming that the thiol-ene reaction is the primary covalent reaction between neighboring particles and intraparticle reactions are negligible.

Rheometry for all the peptides at the different concentrations (Table 1) revealed moduli increases that are not directly proportional to increases in concentration (e.g., modulus at 0.50 g/mL is greater than double the modulus at 0.25 g/mL for all samples). As seen in FIG. 4, the rate of bundlemer crosslinking increases with the concentration, providing insight into network formation. At the highest concentration, the initial bundlemer crosslinking is observed to be translationally diffusion-limited, with limited impact owing to the orientation of bundlemer functional groups. In contrast, at lower concentrations the rate of crosslinking becomes dependent on the number of functional groups and/or bundlemer particle orientation. At lowest concentrations, this effect is even more likely given that more network defects would be present in a more dilute system. Given these observations, the combination of bundlemer diffusion and particle orientation affects network defects leading to a non-linear increase in modulus with concentration.

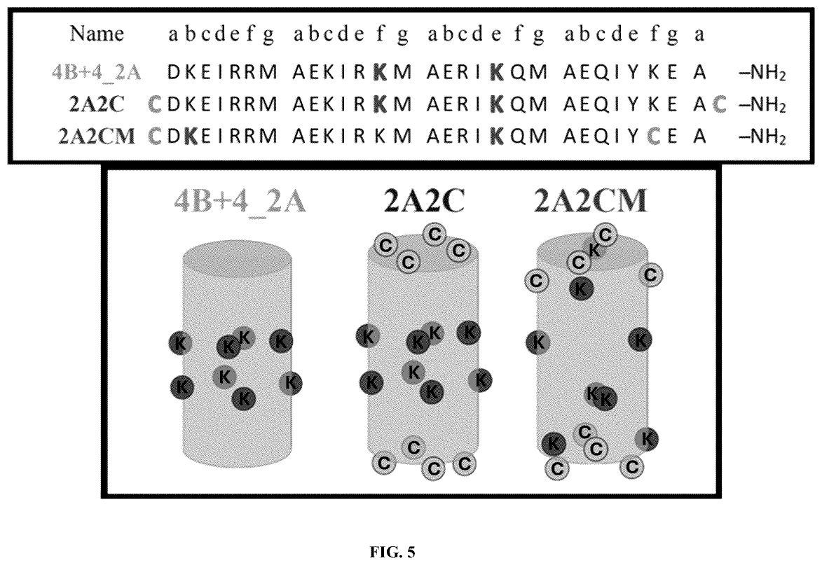

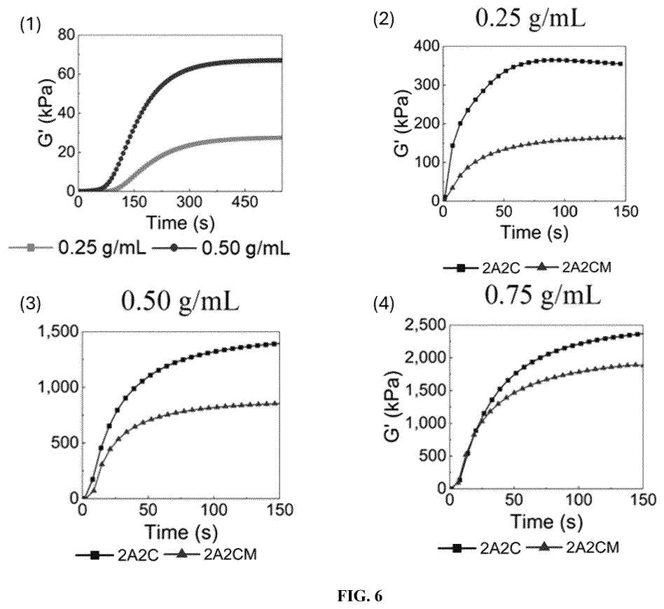

While the increase in the number of crosslinkable sites exhibited a clear effect on the modulus of final networks in FIG. 4, the bundlemer architecture allowed for the specific study of differences in crosslinking site spatial display. Two additional sequences were synthesized that specifically altered the crosslinking sites displayed in bundlemer 2A2C. The first alteration keeps only the alloc side chains (4B+4_2A, see FIG. 5) to directly assess the importance of the thiol group s and the thiol-ene reaction as opposed to the potential of unlikely alloc-alloc radical crosslinking. The second design alteration, designated 2A2CM, contains the same number and type of crosslinkable sites as 2A2C but with slightly altered position within the constituent peptides (see FIG. 5). Importantly, the thiol and alloc groups in the 2A2CM sequence are in closer proximity on the bundlemer surface than the 2A2C sequence, potentially enabling intrabundle crosslinking. FIG. 6 reveals the clear impact these new designs have on the consequent network crosslinking. Despite having an identical number and type of reactive sites, the 2A2C and the 2A2CM sequences displayed significantly different moduli (see Table 2).

TABLE 2
Storage Modulus (kPa) and Standard Error (kPa)
Calculated from Triplicates of Each Sequence
at 0.25 g/mL, 0.50 g/mL, and 0.75 g/mL

Storage Modulus (G′, kPa)

| Concentration | 4B ± 4_2A | 2A2C | 2A2CM |
|---|---|---|---|
| 0.25 g/mL | 23 ± 3 | 340 ± 10 | 170 ± 5 |
| 0.50 g/mL | 55 ± 6 | 1,300 ± 60 | 760 ± 80 |
| 0.75 g/mL | — | 2,300 ± 80 | 1,700 ± 20 |

In the original 2A2C building block, the alloc reactive groups in the center of the bundlemer particles are exposed and able to react easily with terminal cysteines from different bundlemer particles resulting in the formation of significant interbundle crosslink sites and the stiffest networks. In stark contrast, the 2A2CM displays one alloc group near the N-terminus of the peptide close to a terminal cysteine making intrabundle crosslinking possible that will not participate in interbundle network formation. Similarly, one cysteine on each peptide is no longer the C-terminal amino acid but is now several amino acids from the terminus close to an alloc that is near the center of the peptide and particle. The different display caused 2A2CM to have a consistently lower modulus as compared to 2A2C, presumably due to intrabundle crosslinking that can occur in 2A2CM and that does not contribute to the network stiffness.

To further support the hypothesis regarding intrabundle vs interbundle crosslinking for the 2A2CM peptide, two coarse-grained (CG) simulations were conducted where the reactive sites in the CG bundlemers were positioned analogous to those in 2A2C and 2A2CM, respectively. By employing bond creation algorithms, the crosslinking formation process observed in experiments can be mimicked. Using the 0.50 g/mL condition in the simulation, the number of the interbundle crosslinks in 2A2C was observed to be double than observed in 2A2CM. This observation is consistent with the modulus results presented in Table 2 where 2A2C exhibits a modulus approximately double that of 2A2CM. This precise control over chemical crosslinking is often a design challenge in traditional polymer systems. With bundlemer particles, this modified sequence demonstrates the ability of reactive site design to target a specific combination of intra- and interbundle crosslinking in resulting films. The modulus for 4B+4_2A without cysteines/thiols shown in FIG. 6 indicates that alloc-alloc crosslinking results in a drastically lower modulus than observed in the thiol-ene networks (e.g., 23 and 55 kPa for 4B+4_2A at 0.25 and 0.50 g/mL, respectively, vs 340 and 1300 kPa for 2A2C at the same concentrations) (Table 2), showing this crosslinking mechanism does not contribute in a significant way to the ultimate moduli of the thiol-ene films.

The sequence 4B+4_2A with no cysteines for thiol-ene crosslinking and consequent poor network formation displayed unexpected solution behavior, which is much different than the sequences that also contained cysteines. While 0.25 and 0.50 g/mL suspensions were crosslinked and characterized rheologically (see FIG. 6), an attempted 0.75 g/mL sample was unable to be studied due to the formation of a highly turbid, viscous suspension that was not observed in any cysteine-containing samples. To evaluate the cause of the turbidity, transmission electron microscopy (TEM) and small-angle x-ray scattering (SAXS) experiments were conducted with clear evidence of ordered, self-assembled lattice formation in solution. Since the original 4B+4 parent sequence does not exhibit this solution assembly behavior, it is clear the display of the alloc groups around the center of the bundlemer particles drove lattice solution self-assembly. However, two other studied sequences (2A1C and 2A2C) used for the thiol-ene reaction have allocs in the same positions as 4B+4_2A, but they do not exhibit the same lattice self-assembly behavior and only produced amorphous, crosslinked network films. This drastic difference in ordered vs. amorphous nanostructure between almost identical bundlemer sequences is rationalized in the following experiment.

### Experiment 2: Nanostructure Analysis: Physical Lattice Assembly

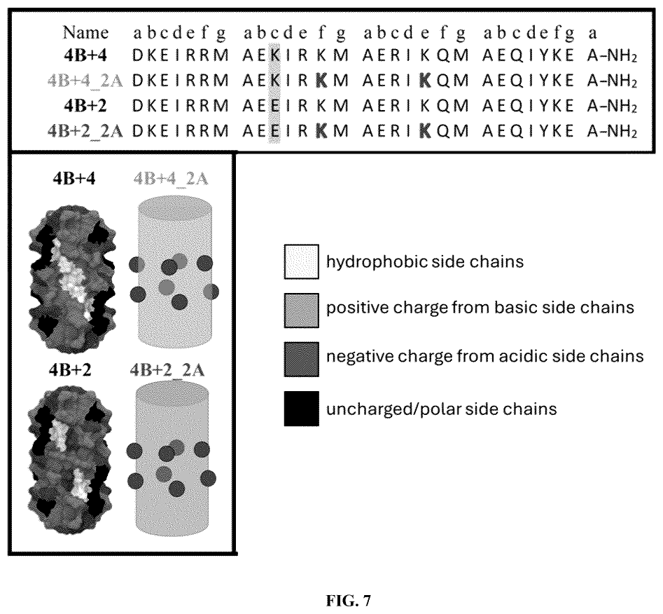

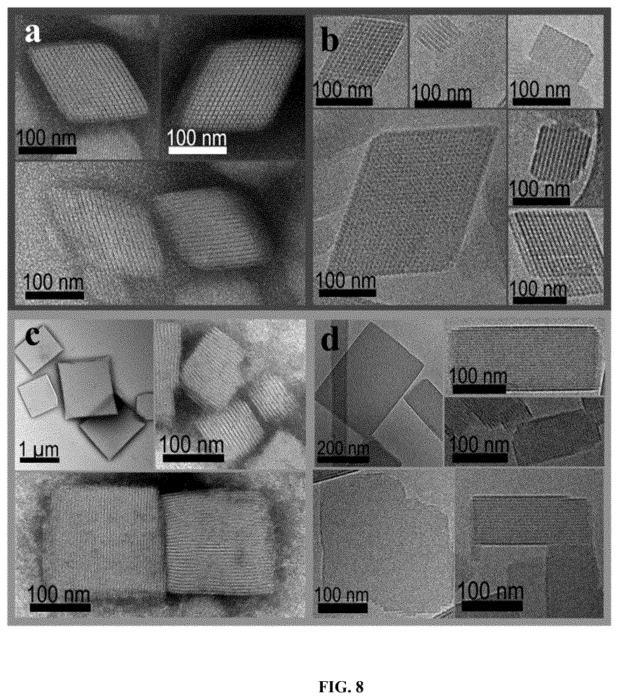

The formation of an unexpected, ordered lattice nanostructure in sequence 4B+4_2A inspired morphological and computational studies to determine the lattice structure and the extent of control thereof through different solution conditions. While the alloc groups present hydrophobic patches on the surface of the bundlemer particles that lead to particle assembly, the lattice structure only was observed in the sequences without cysteines close to the bundlemer termini. Therefore, an additional bundlemer-forming sequence, 4B+2, was targeted for alloc functionalization to observe if lattice formation is possible with other, similar peptide building blocks that do not contain cysteines near the termini. The only difference between 4B+4 and 4B+2 is the net charge at neutral pH (+4 and +2 respectively) due to a substitution of a single amino acid from lysine to glutamic acid in position c of the second heptad. The two modified sequences, 4B+2_2A and 4B+4_2A (see FIG. 7), despite having slightly different amino acid sequences, exhibit similar self-assembly behavior and lattice formation as indicated by SAXS, TEM, and cryoTEM. To study the lattice structures, peptide assembly was observed in both MilliQ and 50 mM phosphate buffer (pH 7). At high concentrations for each peptide, both the water and buffer solutions had the same lattice assemblies despite the different solvent conditions. Representative, negatively stained TEM images and cryo-TEM images for 4B+2_2A and 4B+4_2A are shown in FIG. 8. The microscopy data showed that 4B+2_2A formed overall diamond-shaped particles while the 4B+4_2A formed more rectangular particle morphology, and the addition of salts for both peptides allowed the particles to grow larger than in pure MilliQ. In both TEM and cryo-TEM, one can observe different projections of the same lattice structure in both peptides. To further validate that the alloc functional groups are driving assembly, the unmodified versions of both peptides, 4B+2 and 4B+4, were studied at high concentrations and exhibit only disordered aggregation.

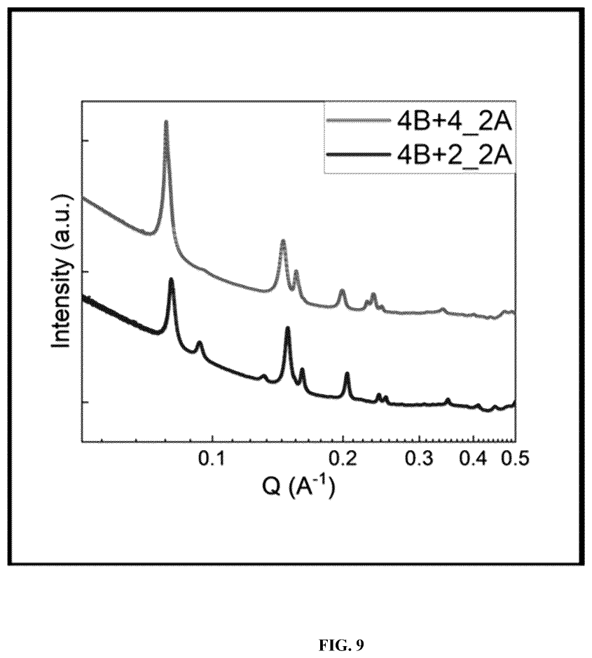

FIG. 9 shows SAXS diffraction data for 4B+4_2A and 4B+2_2A from 5 wt %/vol solutions of lattice particles in 50 mM phosphate buffer. These two sequences displayed similar diffraction peaks, with minor changes in peak intensity and exact peak location between the samples. The peak positions were analyzed by calculating their relative positions using q/q*, where q* is the long spacing peak observed in the SAXS. By comparing the peak ratios, diffraction peaks were directly compared to the relative positions of standard lattice packings, such as body-centered cubic (BCC), face-centered cubic (FCC), and hexagonal closed-packed (HCP) crystal structures. For an FCC lattice, the first five relative positions are q/q*=1, \sqrt{4/3}, \sqrt{8/3}, \sqrt{11/3}, and 2. The relative position of the 4B+2_2A sequence best matched an FCC-type lattice; however, the precise arrangement and orientation of the bundlemers within the lattices was unclear. To further explore the assembled lattice structures, an AI guided approach was developed to rebuild the assembled lattice structure in a physical model by optimization of interbundle distances so that computed structure factors could be matched with experimental SAXS data. Here, a technique similar with that used widely for crystallographic structure elucidation from powder diffraction data, where an optimization algorithm searches for the best matched structures between an evolving model and the experimental diffraction data. This method aims to converge the disparity between experimental observations and theoretical calculations, achieved by iteratively fine-tuning the parameters within the prescribed physical model until the difference is reduced below a predetermined threshold or after a certain number of iterations is reached.

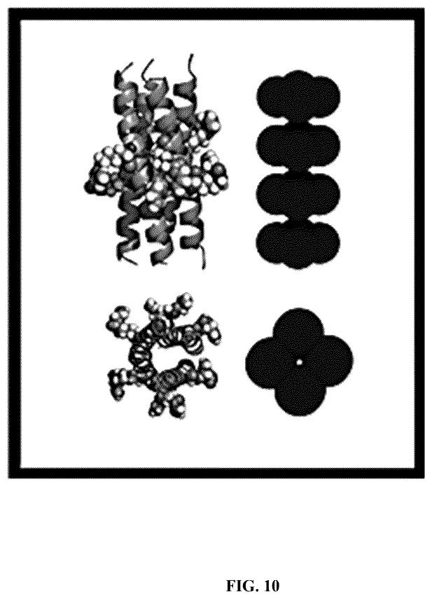

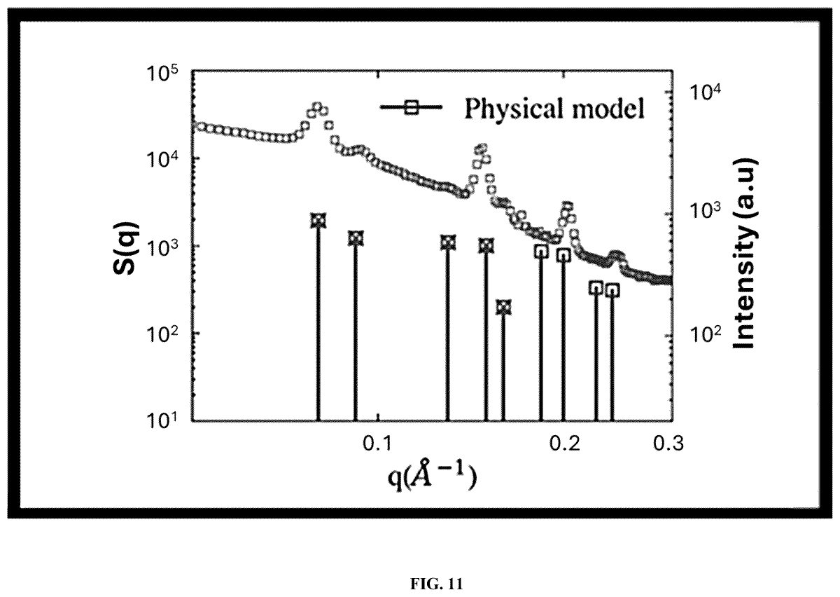

In the physical model, the bundlemers are treated as rigid cylinders and are arranged into a starting structure similar with an FCC truss lattice inside a given box with periodic boundaries as shown in FIG. 10. During the optimization, the orientations of the bundlemers are fixed as cylinders inside the lattice, and only the refinement parameters designed to govern the local, interbundle distance were optimized. After 300 optimization iterations, the improvements in the objective function, which is the mean absolute error (MAE) between peak positions in calculated structures factors vs experimental peak positions in the SAXS results, were minor. The best result well matched the experimental results as indicated by the low MAE of 0.06 nm for the first five peaks, as shown in FIG. 11. Only the first five peaks were matched given the interest in the larger length scale lattice structure and interbundle packing/orientation. Local interactions among specific amino acids and functional groups cannot be captured given the core graining of this model. In addition to matching the SAXS diffraction data well, the calculated lattice structures have reasonable spacings and symmetries that agree well with both TEM and cryoTEM images.

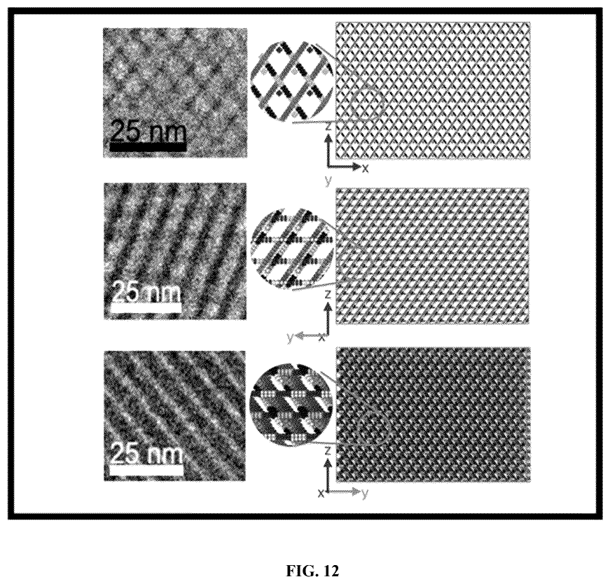

Snapshots obtained from different projections of the calculated physical model (see FIG. 12) exhibit a striking similarity with microscopy results. Examination of the computationally achieved lattice structures revealed that the coarse-grained bundlemers attach in a side-to-side fashion roughly at the positions of the displayed alloc groups, which further supports the role of the alloc group hydrophobic interactions in forming the truss-like FCC lattice structures in these systems. Furthermore, bundlemer particles were observed to form pairwise interactions within the lattice instead of three or more bundlemers clustering together, with each bundlemer having only one attached neighbor. This pairwise interaction could result from the concentration of alloc groups on two sides of the bundlemer as shown in FIG. 10 in the illustration generated from an all-atom simulation.

When two bundlemers attach during solution assembly, the alloc groups become saturated with respect to hydrophobic interaction, and there is no space for other alloc groups from other bundlemers to fit. With this hypothesis, all-atom simulations were conducted to analyze interparticle angles and local alloc group interactions. Inhomogeneous alloc group distribution was observed from the surface of single bundlemer particles. Clusters of alloc groups were present on two specific sides of the bundlemer with each cluster comprised of two alloc groups. In all-atom simulations of the interaction between two bundlemer particles, these alloc clusters exhibited a marked affinity towards the alloc clusters of the neighboring bundlemer, which likely served as the primary active site facilitating interparticle assembly. Additionally, the angles between the two bundlemer particles ranged from 60 to 70 degrees, consistent with the structures proposed from coarse-grained modeling in FIG. 12. Overall, with the use of this AI guided approach, the interparticle structure of the peptide lattice was successfully estimated providing a physical picture of how the bundlemers interact with each other on the nanometer length scale. The display of the hydrophobic alloc side groups resulted in discovery of a new truss-like, porous FCC lattice structure, that had not previously been observed in solution assembled peptide systems.

### Conclusions of Example 1

Bundlemer peptides were selectively modified with alloc protected lysine for applications in covalent networks and physical lattice assemblies. Modified peptides demonstrated success in the ability to create controllable crosslinks in peptide networks, and drive assembly of a physical lattice via hydrophobic interactions. By tuning the display of chemical functional groups on the bundlemer particle exterior with protein-like specificity, the degree of intra-vs. interbundle crosslinking was manipulatable and allowed for improvement in the mechanical performance of the ultimate crosslinked, hydrated films. This purposeful manipulation of crosslinking orientation and material properties is a common challenge in polymer networks and highlights the opportunity made possible with the bundlemers disclosed herein for use in highly structured network materials in the future. When bundlemer particles contained alloc groups displayed around the center of the monomer, the particles formed an intricate, porous lattice with approximate FCC packing. Using this study as an inspiration for future material design, the formation of tunable and controllable bundlemer networks with high levels of order on the molecular level via the computational design of targeted, ordered nanostructures with specific display of reactive sites for covalent crosslinking and physical interactions is achievable.

### Example 2: Peptide Bundlemer Networks Driven by Natural and Non-natural

### Hydrophobic Side Chains

Materials and Methods used in Example 2

### Materials

All reagents were purchased from Fisher Scientific, unless otherwise specified, and used as received. Milli-Q water, used in synthetic and assembly procedures, was obtained from a milli-pore ultrafiltration system (approximate resistance of 18.2 mΩ·cm² at 23° C.).

### Solid-Phase Peptide Synthesis (SPPS)

Automated, microwave assisted solid-phase peptide synthesis (SPPS) was performed using a CEM Liberty Blue. All syntheses were performed at 0.25 mmol scale using rink amide resin (loading between 0.3-0.6 mmol/g). Standard fluorenylmethoxycarbonyl (Fmoc) chemistries were employed. Fmoc-protected amino acids (ChemPep) were prepared in N,N-dimethylformamide (DMF) at a concentration of 0.2 M. ethyl cyano (hydroxyamino)acetate (Oxyma®Pure, CEM, 4 eq.) and N,N-diisopropylcarbodiimide (ChemImpex, 8 eq.) coupling reagents were prepared at a concentration of 1 M in DMF. Double coupling of amino acid residues was conducted at 90° C. for 4 minute cycle times, followed by 4 minute Fmoc deprotection cycles at 75° C. using a solution of 20% (v/v) piperidine in DMF. For peptide syntheses requiring post-synthetic modifications, Boc-Asp (OtBu)—OH (AAPPTec) was used as the N-terminal amino acid (replacing Fmoc-Asp (OtBu)—OH) for protection of the termini in basic conditions.

### Synthesis of Non-Natural Amino Acid Fmoc-Lys (Furan)-OH·HCl

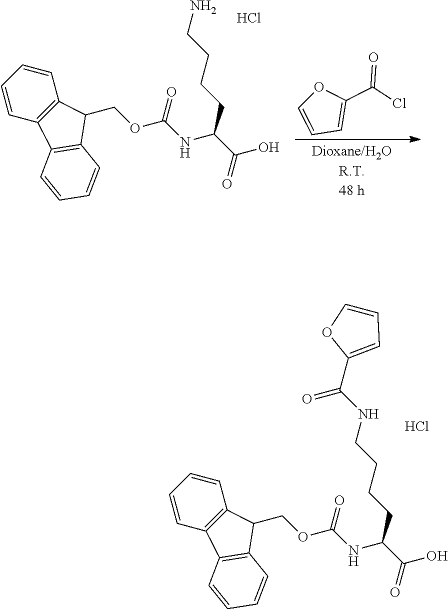 [SMILES: NCCCC[C@H](NC(=O)OCC1c2ccccc2-c2ccccc21)C(=O)O ; Cl ; O=C(Cl)c1ccco1 ; O=C(N[C@@H](CCCCNC(=O)c1ccco1)C(=O)O)OCC1c2ccccc2-c2ccccc21 ; Cl]

The synthesis of the non-natural amino acid, Fmoc-Lys (Furan)-OH·HCl (ChemPep, 99.3%), was adapted from a previous report (see D. Nielsen et al., Protein and peptide letters 23 (9): 772-776). Briefly, Fmoc-Lysine-OH·HCl (3.32 g, 8.19 mmol) was dissolved in a mixture of 33 mL of 1,4-dioxane (TCI America, >99%) and 33 mL of an aqueous solution of 25% (w/v) potassium carbonate at room temperature. Following, a solution of 2-furoyl chloride (1.06 g, 8.13 mmol) in 33 mL of 1,4-dioxane was added dropwise to the solution of Fmoc-Lysine-OH·HCl. After complete addition of 2-furoyl chloride, the reaction was allowed to proceed at room temperature for 48 hours and monitored using thin layer chromatography. Upon completion, the reaction solution was diluted with 180 mL of DI water and washed once with tert butyl methyl ether. The aqueous layer was recovered and acidified using concentrated HCl. Upon acidifying, a yellow precipitate formed, and the aqueous layer was extracted against dichloromethane and concentrated in vacuo to yield the final product as a light yellow solid (3.57 g, yield: 95%). The newly synthesized Fmoc-Lys (Furan)-OH·HCl was used without further purification in automated solid-phase peptide syntheses to produce the peptide +4K Furan13, 19.1H-NMR (400 MHZ, DMSO-d6): δ (ppm) 8.34 (t, J=5.8 Hz, 1H), 7.89 (d, J=7.5 Hz, 2H), 7.79 (d, J=1.2 Hz, 1H), 7.72 (d, J=7.5 Hz, 2H), 7.62 (d, J=8.0 Hz, 1H), 7.41 (t, J=7.4 Hz, 2H), 7.32 (t, J=7.4 Hz, 2H), 7.05 (d, J=3.4, 0.8 Hz, 1H), 6.59 (dd, J=3.5, 1.7 Hz, 1H), 4.24-4.18 (m, 3H), 3.91-3.87 (td, 1H), 3.19 (q, J=6.8 Hz, 2H), 1.74-1.30 (m, 6H). UPLC/ESI-MS: calculated: 462.5 Da; found: 463.4 Da [M+H]+ and 925.7 Da (self-adduct, [M+M]2+).

### Site-Specific Non-Natural Amino Acid Modification

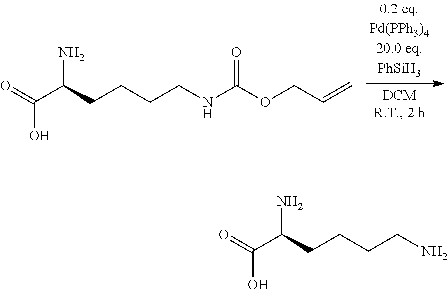 [SMILES: C=CCOC(=O)NCCCC[C@H](N)C(=O)O ; NCCCC[C@H](N)C(=O)O]

Selective deprotection of alloc-protected lysine side chains was carried out using a catalytic amount of tetrakis(triphenylphosphine) palladium (0) (Pd(PPh₃)₄, Chemlmpex, ≥99.5%) (0.2 eq., 0.5 mmol, 58 mg) in the presence of phenylsilane (PhSiH3, ThermoScientific, 97%) (20 eq., 5.0 mmol, 617 uL) and dichloromethane (DCM) at room temperature for 2 h with constant mixing. After 2 h of reacting, the deprotection solution was drained and the peptidyl resin was washed several times with fresh DCM and methanol (MeOH). Successful removal of the alloc protecting group to reveal free amines was confirmed via Waters XEVO G2-XS Qtof ultra-performance liquid chromatography electro-spray ionization mass spectroscopy (UPLC/ESI-MS).

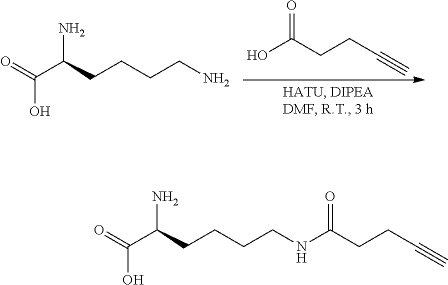

For coupling of the alkyne group to the lysine side chain, 4-pentynoic acid (5 eq., 1.25 mmol, CombiBlocks, 97%) was combined with hexafluorophosphate azabenzotriazole tetramethyl uronium (HATU) (5 eq., 1.25 mmol, CombiBlocks, 97%) in 7.4 mL of DMF. To this mixture, N,N-diisopropylethylamine (DIPEA, TCI America) (10 eq., 2.50 mmol) was added, and the reaction mixture was mixed at room temperature for five minutes. The alkyne reaction solution was then added directly to alloc deprotected peptidyl resin, and the reaction was allowed to proceed for at least 3 h at room temperature with constant mixing. After 3 h, the reaction mixture was drained, and the resin was washed several times with fresh DMF. Successful coupling of the alkyne to lysine side chains was confirmed via UPLC/ESI-MS.

### Peptide Cleavage

Peptidyl resin was prepared for cleavage by washing once with a solution of 10% acetic acid in DCM, followed by three washes of DCM, two washes of MeOH, and three washes of DCM. Peptides were cleaved from resin using a solution of 95/2.5/2.5 trifluoroacetic acid (TFA)/Milli-Q water/triisopropylsilane (TIPS) (v/v/v) with phenol and 1,4-dithiothreitol (DTT) (50 mg/mL) as additional scavenging agents. The cleavage reaction proceeded at room temperature for 3 hours with constant mixing. Following cleavage, the crude peptide solution was collected and precipitated dropwise into 40 mL of cold diethyl ether. The crude product was centrifuged at 4° C. at 4.4K RPM for 10 minutes. After initial centrifugation, the supernatant was discarded, and the crude peptide was resuspended in fresh ether and subjected to centrifugation at 4° C. at 4.4K RPM for 5 minutes. The ether wash steps were repeated two more times. The crude peptide was allowed to dry at room temperature under a stream of nitrogen prior to purification.

### Peptide Purification

Peptide purification was performed using reversed-phase high pressure liquid chromatography (RP-HPLC). Peptides were purified using a Waters 2535 Quaternary Gradient Module HPLC equipped with a Waters 2489 UV/Vis detector (dual wavelength monitoring of 214 and 280 nm). Preparative scale purification was carried out using a Waters XBridge C18 column (5 μm, 30×250 mm). Selected mobile phase compositions consisted of water and acetonitrile modified with 0.04% (v/v) trifluoroacetic acid. Crude peptide solutions were prepared for RP-HPLC at a concentration of 20 mg/mL and purified using a focused gradient of 25 to 55% acetonitrile. Peptide purity and molecular weight were determined using UPLC/ESI-MS.

### Circular Dichroism (CD) Spectroscopy

CD spectroscopy measurements were performed using a JASCO J-1500 CD spectrophotometer. Solutions of peptides were prepared at 0.10 mM in milli-Q water titrated to pH 7 (solubilizing conditions equivalent to SAXS and TEM experiments). Samples were analyzed in CD using 1 mm pathlength quartz cuvettes from 185-250 nm at a scanning rate of 50 nm/min. A digital integration time (D.I.T) of 4 seconds was selected to maximize signal to noise ratio across the investigated wavelengths. Data was normalized by number of residues using the equation MRE=CD/(c*l*n), where MRE is mean residue ellipticity (deg cm²dmol⁻¹), CD is the signal intensity output from experiments (mdeg), c is concentration (M), l is cuvette pathlength (mm), and n is number of residues.

### Small Angle X-Ray Scattering (SAXS)

Samples were prepared at 5 w/v % and transferred to quartz capillaries with a wall thickness of 0.01 mm and outer diameter of 1.5 mm (Charles Supper Company). Measurements were performed at Brookhaven National Laboratory NSLS-II 16-ID (LiX) beamline with a beam energy of 15.3 keV (0.81 Å). Exposure time was 0.5 s for each sample with data averaged over 20 frames.

Transmission Electron Microscopy (TEM) and Cryogenic TEM (cryoTEM)

TEM imaging was conducted on a FEI TALOS F200C TEM with an accelerating voltage of 200 keV. Samples were prepped on 200 or 400 mesh ultra-thin carbon film copper grids (Electron Microscopy Sciences). Samples were prepared by glow-discharge coating grids for 30 seconds using a PELCO easiGlow™ 91000 glow discharge cleaning system. Samples were then cast on the grid (5 uL) for 30-60 s and excess solution wicked away using a KimWipe. Samples were stained using 5 uL of 2 w/v % phosphotungstic acid solution (titrated to pH 7 using 1M NaOH) with deposition time ranging from 30-50 s. Grids were dried at room temperature for at least 10 minutes before imaging. CryoTEM was conducted on a Talos TEM using an accelerating voltage of 200 keV. Lacey Carbon TypeC only 400 mesh grids (Ted Pella Inc.) were glow discharged coated for 30 seconds. Samples were prepared using a Vitrobot with a 100% humidity chamber with varied deposition/blotting settings. Samples were vitrified in liquid ethane and transferred to liquid nitrogen bath prior to imaging.

### Molecular Dynamics (MD) Simulations

All-atom simulations using NAMD2 for both monomer and dimer system were performed to gain insight on the structures and fluctuations of the bundlemers. The CHARMM36 force field was used to parameterize the system, and the alkyne group was modeled using the CHARMM36 general force field (e.g., see Mackerell, A. D. et al., Biopolymers 56, 257 265 (2000); Vanommeslaeghe, K. et al, J Comput Chem 31, 671 690 (2010); Vanommeslaeghe, K. & MacKerell, J. A. D, J Chem Inf Model 52, 3144 3154 (2012), Yu, W. et al., J Comput Chem 33, 2451 2468 (2012); Croitoru, A. et al., J. Chem. Theory Comput. 17, 3554-3570 (2021)). For the monomer simulation, the bundlemer was put in the center of a cubic box with edge length of 60 Å and TIP-3P water molecules at liquid density. Periodic boundary conditions were applied. Overlapping waters were removed. Ionization states of the amino acid termini and side chains were chosen to be those expected at pH 7. Na⁺ and Cl⁻ were added to the system to balance electrostatic charges, ensuring no net overall charge, in a manner consistent with [NaCl]=0.15 M. An energy minimization using steepest descent of 100,000 steps was performed, and then the system was simulated in the NVT ensemble with T=400 K for 10 ns, controlled by Nosé-Hoover Langevin thermostat. Subsequently, the temperature was decreased to 300 K, and the simulation was run for 190 ns. VMD and PyMOL were used for visualization. During the entire simulation, the bundlemer maintained the tetrahelical structure. The final 150 ns was sampled and used for analysis.

For the dimer simulations, the two bundles were initially parallel and the distance between their centers was 30 Å. A similar simulation procedure was carried out, except that the rectangular simulation box was now a 100 Å×70 Å×70 Å parallelepiped, and the system was simulated in an NPT ensemble with p=1 atm and T=300 K for 200 ns. The constant pressure was controlled by Nosé-Hoover Langevin barostat. Among the systems tested, only the configuration with the alkyne groups at site 13 facing those at site 19 formed a stable contact structure, while in the others the two bundlemers eventually dissociated. In this stable dimer system, a persistent close contact between the alkyne groups at sites 13 and 19 was maintained throughout the simulation. The angles between the major (superhelical) axis of the two bundles were calculated. The hydrophobic interaction between the alkyne groups allows one bundle to readily rotate with respect to the other while the bundles remain in proximity.

Lattice structures were elucidated through computational modeling as previously established in earlier studies (see Tian, Y., Zhang et al., Org Biomol Chem, 15 (29), 6109-6118; and Sinha, N. J. et al., Physical Review Materials, 5 (9)). Each bundlemer was treated as a rigid cylinder systematically placed in a box, and Bayesian Optimization (BO) was employed to refine cylinder positions and unit cell parameters so that the calculated structure factor aligned with the first three peaks in the experimental SAXS data. The initial unit cell, serving as the building block of the models, includes two parallel bundlemers. Six refinement parameters were used and involved three-dimensional coordinates (x_{d}, y_{d}, z_{d}), specifying the relative displacement between the bundlemer dimers, as well as the dimensions of the unit cell (xᵤ, yᵤ, zᵤ). To make the system symmetric, one constraint was set with xᵤ=2x_{d}. Once the initial unit cell was built, it was replicated six times along each Cartesian dimension, using the parameters xᵤ, yᵤ, zᵤ. In the optimization over the dimer and unit cell parameters was run for 300 steps. This procedure was sufficient to obtain a mean absolute error (MAE) less than 0.016 nm⁻¹ for the sum over the differences in the q values corresponding to the first three peaks of the calculated and experimental SAXS structure factors. The best results with lowest MAE were x₁=2.64 nm, y₁=0.0 nm, z₁=0.0 nm, xᵤ=5.28 nm, yᵤ=2.64 nm, zᵤ=4.18 nm. The computational reconstruction presented here focused solely on the geometric arrangement of the bundlemers based on structure factor calculations to match the experimental data.

### Experiments and Results of Example 2

### Experiment 1: Sequence Modifications and Characterization

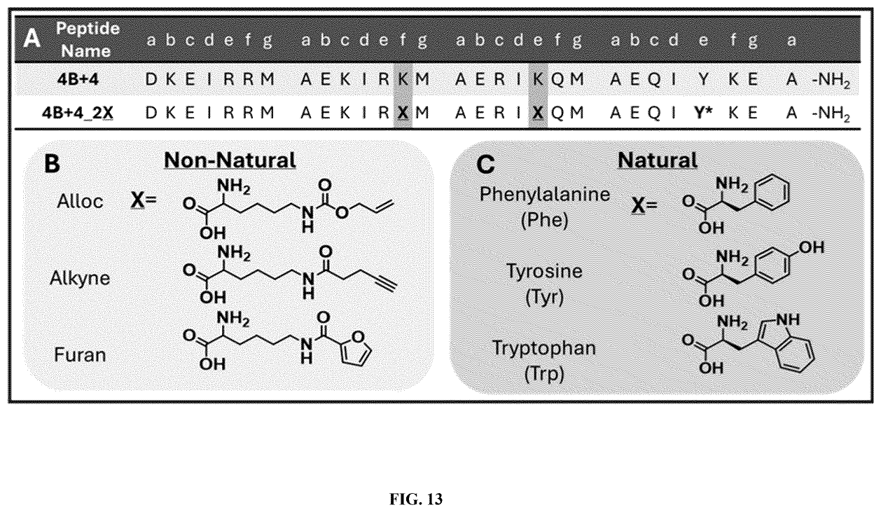

The 4B+4 sequence, named to indicate the sequence creates a tetrameric peptide bundlemer where each peptide possesses a net charge of +4, was selected as the parent sequence for hydrophobic side chain modification (sequence depicted in FIG. 13). In Example 1 this sequence was engineered with two alloc-protected lysines (K13 and K19, highlighted in FIG. 13), resulting in the formation of coiled coil bundlemer particles with eight alloc groups displayed from the center of the particle. These particles subsequently assembled into an FCC-truss-like, porous lattice.

In this experiment, five new hydrophobic side chains, derived from both non-natural lysine modifications (e.g., furan and alkyne) and natural aromatic amino acids (phenylalanine, tyrosine, and tryptophan), were investigated to assess their impact on porous lattice formation. These functional groups were strategically selected based on their potential to promote self-assembly through hydrophobic and π-π interactions, as well as their potential use in covalent conjugation reactions. The sequences studied (outlined in FIG. 13) feature hydrophobic modifications at amino acid positions 13 and 19 (shown as X in sequence 4B+4_2X and illustrated in FIG. 13). The non-natural furan and alkyne moieties were chosen due to their hydrophobic nature, ease of incorporation, and potential biorthogonal reactions (e.g., the Diels-Alder cycloaddition and copper (I) catalyzed azide-alkyne cycloaddition (CuAAC), respectively); these modifications also provide valuable insight for design of bundlemer-forming sequences that incorporate non-natural amino acid side chains. Upon coupling these functional groups to existing lysines, the resultant modified side chains are expected to exhibit comparable flexibility to the alloc-protected lysine bundlemers evaluated in Example 1. The natural amino acids phenylalanine, tyrosine, and tryptophan were chosen to investigate the capacity of hydrophobic aromatic functional groups to drive lattice self-assembly, with all three amino acids having aromatic side chains that also can form π-π interactions. These amino acids are particularly advantageous for both recombinant expression strategies and covalent crosslinking applications, given their natural occurrence and intrinsic chemical reactivity.

Circular dichroism (CD) spectroscopy confirmed the formation of coiled-coil bundlemer particles following site-specific modification. The characteristic alpha-helical minima at 208 and 222 nm were observed, and the ratio of absorbances at 208 nm and 222 nm were close to unity, indicative of stable coiled coil structures. These results support the ability to selectively modify the bundlemer-forming peptides with a range of hydrophobic functional groups without compromising subsequent coiled-coil assembly. This modularity is attributed to the computationally optimized design with the intrinsic stability imparted by the hydrophobic core of the original parent bundlemer.

### Experiment 2: Lattice Formation and Characterization

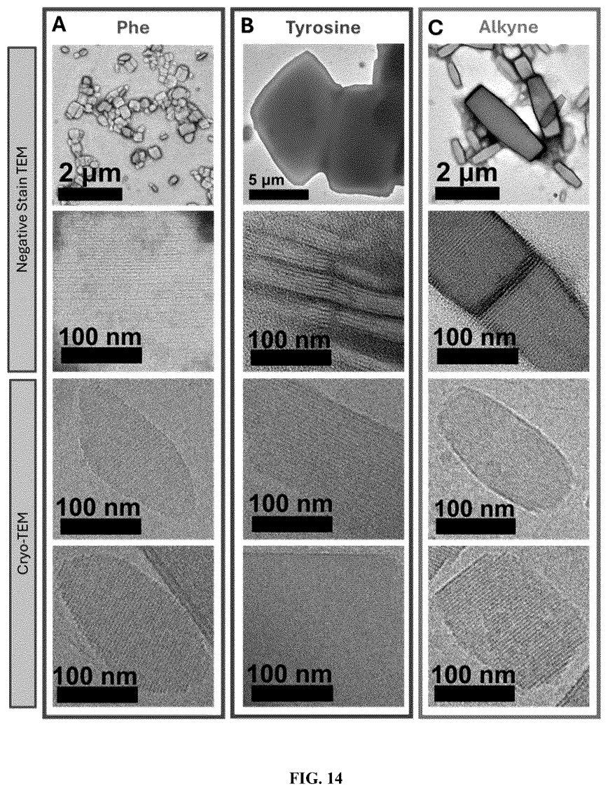

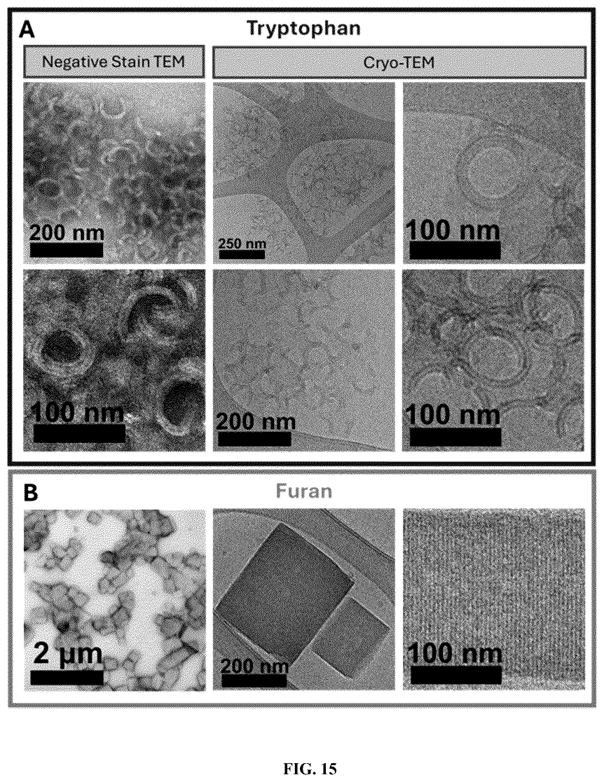

The assembly of the modified sequences in solution was examined by dissolving the peptides in Milli-Q water (titrated to pH 7). Under these conditions, each sequence immediately formed turbid solutions. All samples were subsequently analyzed using TEM and cryoTEM, with structural observations summarized in FIG. 14 and FIG. 15. Negative-stain TEM images revealed effective penetration of the stain into the lattice pores, clearly highlighting lattice porosity. In complementary cryoTEM imaging, the contrast between electron-dense, peptide-rich regions and lighter solvent-filled pores further confirmed the porosity of these nanostructures. All five modified sequences exhibited ordered lattice self-assembly; however, the resulting nanostructures were notably different from the alloc-driven assemblies of Example 1. Specifically, peptides modified with alkyne, furan, tyrosine, and phenylalanine assembled into porous, layered lattices with approximate pore dimensions of 2-3 nm (FIG. 14 and FIG. 15). In contrast, the peptide modified with tryptophan formed rounded nanostructures, resembling nanotubes with an outer diameter of approximately 10 nm (FIG. 15). Given that these particles all share the same parent sequence, these differences underscore how specific chemical functionalities displayed at positions 13 and 19 directly influence porosity and structural morphology.

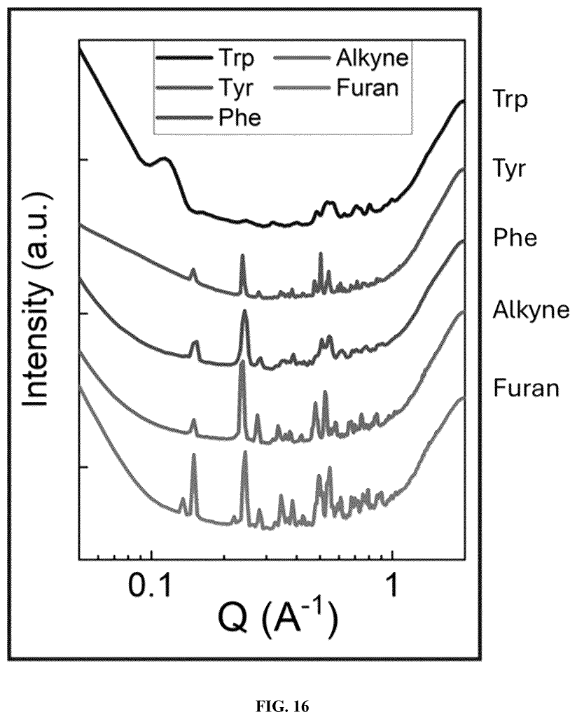

The structures of the lattice nanostructures were further investigated in concentrated solutions (5 w/v %) using SAXS (see FIG. 16). Despite having different hydrophobic side chains, the tyrosine, phenylalanine, and alkyne lattices exhibited similar structure factor peaks, suggesting a similar bundlemer packing arrangement within their respective lattices. The furan-modified bundlemer lattices displayed differences from the nanostructures formed by the tyrosine, phenylalanine, and alkyne-modified bundlemers as indicated by additional scattering peaks (see FIG. 16). The nanostructure of the tryptophan-modified bundlemers assembled into a distinct, rounded particle morphology, evidence of which is clearly observed in the TEM (see FIG. 15) and in the unique SAXS structure factor signature (see FIG. 16). These observations collectively confirm the fundamentally different mode of self-assembly for the tryptophan-based bundlemers. In comparison to the alloc-protected lysine lattices of Example 1, all five new sequences form nanostructures that differ from the Example 1 lattices. This difference is particularly noteworthy for the bundlemers possessing the furan and alkyne non-natural side chains, despite them being amide-conjugated to lysine side chains as in the alloc-functionalized bundlemers of Example 1. The results for the furan- and alkyne-based bundles suggest that despite the comparable side-chain flexibility, the specific local interactions of these functional groups significantly influence the interbundle interactions that direct porous lattice formation.

While the non-natural amino acid side chains resulted in nanostructures that were distinctly different from each other and from the alloc lattices of Example 1, the natural aromatic amino acid side chains of tyrosine and phenylalanine exhibited notably similar assembly behavior. Tyrosine and phenylalanine, which differ only by a hydroxyl group, formed lattice structures nearly indistinguishable by SAXS. Interestingly, these lattices closely resemble the alkyne lattice, despite the aromatic side chains being significantly shorter in length than the alkyne modified lysines. In contrast, the tryptophan-containing bundlemers produced a completely different structure.

### Experiment 3: Atomistic and Dimerization Models

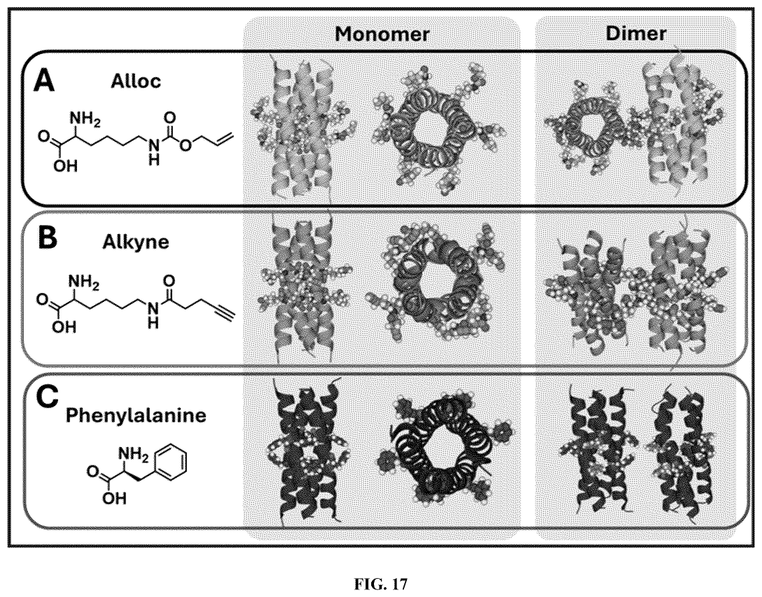

Atomistic modeling and simulations were performed to better understand the structures and molecular interactions associated with the bundlemers and their assembly. The sequences differ in the side chains present at two positions (13 and 19, see FIG. 13). To explore these differences, bundlemers (homotetramers) bearing lysine-alloc, lysine-alkyne, and phenylalanine side chains were modeled and simulated. The lysine-alloc variant forms a lattice that is distinct from that of the lysine-alkyne and phenylalanine variants (see FIG. 14 and FIG. 16). Atomistic simulations of single bundlemers and dimers of bundlemers in the presence of explicit solvent and counter ions were developed as described previously (see FIG. 17; and McCahill, A. L. et al., Peptide Bundlemer Networks or Lattices: Controlling Cross-Linking and Self-Assembly Using Protein-like Display of Chemistry. ACS Nano 2024).

The simulations of the single bundlemers highlight key features and intra-bundle interactions involving the alloc, alkyne, and phenylalanine variants. In each case the eight sites, the 13 and 19 positions in each of the four peptides of the tetramer, form a band of hydrophobic side chains around the center of the cylindrical bundlemer (see FIG. 17). The structure and flexibility of this band of hydrophobic residues is sensitive to the moieties that are displayed. While the alloc variants have the termini of their side chains in compact configuration near the helical backbone, the akyne groups exhibited a more extended, flexible display that is distal from the tetrahelical cylinder. Due to their shorter lengths, phenylalanine side chains took on conformations that are much closer to the bundlemer core, and similar features are observed for tyrosine and tryptophan. These natural aromatic side chains at the 19^{th} position are tightly clustered, and their shorter lengths prevented intra-bundle and intra-helix π-π interactions. The presentation of hydrophobic groups is distinct for each of three side chains: the alloc groups formed compact protrusions from the bundle; the alkyne groups formed extended conformations that are flexible and can potentially readjust to accommodate compact packing of bundlemers; and the natural aromatic side chains presented relatively few side-chain conformations close to the bundle backbone.

Simulations of dimers of bundlemers were used to compare the conformations of alloc-, alkyne-, and phenylalanine-modified variants (see FIG. 17). Distinct differences in the preferred angles between the superhelical axes of the two bundles were observed. Alloc-modified bundlemers predominately formed a nearly perpendicular configuration that is mediated by strong inter-bundle interactions primarily involving the side chains at position 19. In contrast, the alkyne-modified bundlemers preferred a nearly parallel stacking arrangement, dominated by side-chain interactions involving position 19 on one bundlemer and position 13 on the neighboring bundlemer.

Variation in interbundle interactions influenced interparticle dimer structures and subsequent lattice growth. Alloc bundlemer dimer assemblies appeared to involve mediating interactions via position 19, arranged on opposite sides of the bundlemer periphery. On the other hand, alkyne assemblies involved noncovalent, hydrophobic interactions at both the 13 and 19 positions. Furthermore, the alkyne-mediated dimer was observed to be more flexible, with a greater range of angles between the superhelical axes of the two bundles. This increased flexibility of both side chains and the relative orientation of two adjacent bundles likely accommodated the tighter lattice packing and smaller pore sizes (about 2 nm) observed experimentally. This small pore size is consistent with parallel rods arranged with a square symmetry. By contrast, the alloc-bundlemer yielded more intricate lattice architectures with perpendicular pairwise interactions leading to the formation of a truss-like lattice, having larger (about 6 nm) pores.

The phenylalanine-containing bundlemer dimers presented shorter hydrophobic, aromatic side chains, which could impede direct π-π interactions as a result of steric constraints due to other exterior amino acids and the high surface charge of the bundlemers. Simulations revealed a preferential interaction between the phenylalanine side chains and arginine residues on the neighboring bundlemer (aromatic-cation interactions), promoting a parallel alignment reminiscent of that observed in alkyne bundlemer dimers (see FIG. 17). Thus, despite their chemical differences, phenylalanine and alkyne modifications are each compatible with comparable lattice morphology, mediated by inter-bundlemer interactions involving the central, exterior hydrophobic residues.

### Experiment 4: Coarse-Grained Lattice Simulations

Molecular modeling and coarse-grained fits were conducted based on SAXS data obtained from lattices to help elucidate the specific nanostructure of the matching porous lattices formed by alkyne-, phenylalanine-, and tyrosine-modified bundlemers. These methods utilized previously described methods to fit experimental SAXS data with coarse-grained representations (e.g., see Shi, Y. et al., Ordered assemblies of peptide nanoparticles with only positive charge. Nat Commun 2024, 15 (1), 10057; and McCahill, A. L. et al., Peptide Bundlemer Networks or Lattices: Controlling Cross-Linking and Self-Assembly Using Protein-like Display of Chemistry. ACS Nano 2024). Example 1 demonstrated that alloc-functionalized bundlemer dimers adopt an approximate perpendicular orientation, and this orientation is consistent with forming a truss-like FCC lattice that maintains this dimer orientation within the porous structure.

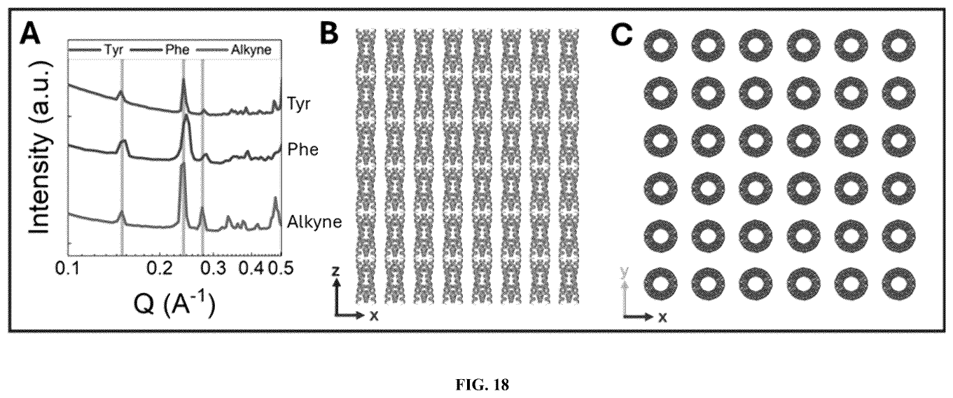

For the alkyne modified peptide bundlemer, the model was obtained by fitting the first three experimental SAXS structure factor peaks for (see FIG. 18). A distinct feature of this lattice packing model is the parallel alignment of bundlemers within the lattice, similar to the orientation observed in the interparticle dimer models. Additionally, bundlemer end-to-end interactions are observed in the alkyne porous bundlemer assemblies, a behavior also observed with alloc-based assemblies. The side-to-side alignment (see FIG. 18) closely resembles that identified in the atomistic alkyne dimer models. The hydrophobic side-chains appeared to facilitate nearly parallel alignment. Given that their structure factor peaks in SAXS occur at the same q values, this fitted lattice model was also applicable to tyrosine- and phenylalanine-modified bundlemers.

### Discussion and Hypothesis for Differences in Assemblies

Without being bound to any particular theory, though the simulations clearly demonstrated specific preferential display and interparticle interactions for alkyne-modified side chains, the behavior of furan-modified sequences proved significantly more complex. Atomistic modeling and dimerization models were conducted for the furan-modified sequences, however, no preferred dimer configuration was observed. This suggests that both the display of side chain conformation and interbundlemer interactions involving the furan side chains are less defined or potentially require a more complex stabilization mechanism than those observed for the alkyne variant. An alternative possibility is that the furan-modified sequences may form multiple distinct lattice structures simultaneously, combining end-to-end stacking (as observed with the other bundlemers) with varied angles and orientations for side-to-side interactions. Such complexity makes it difficult to deconvolute the structure factor peaks and assign them to a specific lattice structure. While no precise structural model currently exists for furan lattices, the observation that they share three major SAXS peaks with alkyne-, phenylalanine-, and tyrosine-bundlemer lattices suggests that at least one of the furan particle configurations may match one of these more defined lattice structures.

The investigation of natural aromatic amino acid side chains has highlighted additional complexity arising from shorter length hydrophobic side chains. Phenylalanine- and tyrosine-modified bundlemers, despite side-chain structural differences compared to alkyne-modified bundlemers, form lattices with nearly identical SAXS profiles, indicating similar coarse-grained lattice structures. For these lattices to match structurally, both the intra- and inter-bundlemer interactions must be localized similarly on the bundlemer surface. Given the shorter length of the natural aromatic side chains and the relatively large distance between particles observed in the proposed lattice (see FIG. 18), additional side chains in addition to those at positions 13 and 19 are likely involved in stabilizing these lattice structures.

The tryptophan-modified bundlemers formed a distinctly different nanostructure, i.e., short, curved nanotubes rather than lattices. The tryptophan-based bundlemer nanotubes exhibited clear curvature, possibly due to steric hindrance from the four centrally positioned tryptophan side-chains. Steric hindrance could influence intra- and inter-bundlemer interactions, resulting in in curved tube structures observed in TEM. This structural frustration could explain the observed incomplete or fragmented nanotubes. Angular offsets between interacting particles could also cause of nanotube formation. Thus, the curvature observed in tryptophan-bundlemer nanotubes could also result from competing end-to-end and side-to-side interactions, combined with subtle angular offset at the inter-bundlemer interface.

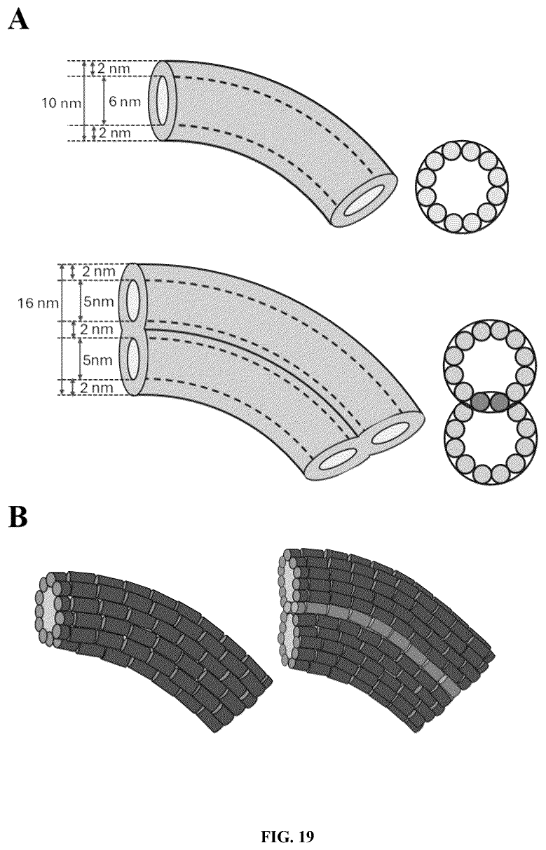

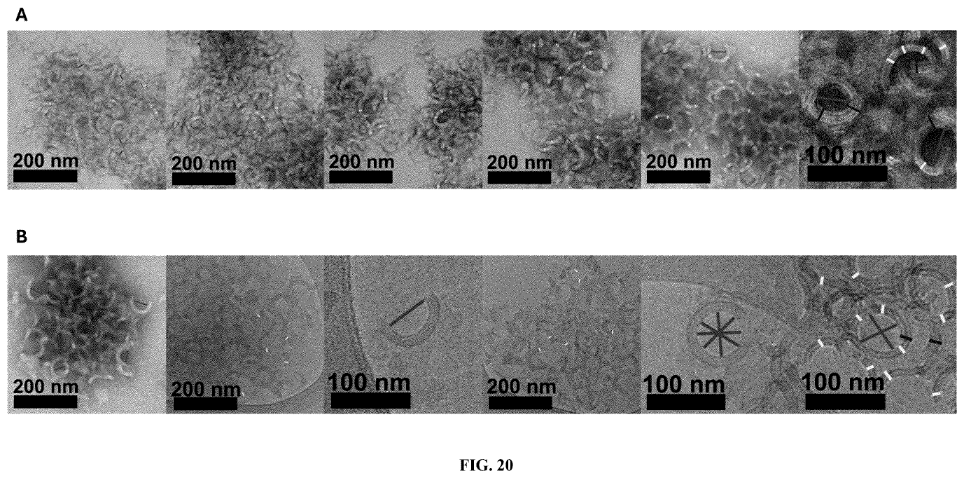

A possible model of the tryptophan-bundlemer assemblies is proposed in FIG. 19, based on bundlemer dimensions, tube curvature, and contrast measurements from TEM and cryoTEM (see Table 3 and FIG. 20), as well as knowledge of bundlemer solution behavior. Measurements were made in ImageJ.

TABLE 3
Statistics for Measuring Distances for Tryptophan Assemblies.

| | Negative Stain TEM | | | CryoTEM | | |
|---|---|---|---|---|---|---|
| | Number | | Std | Number | | Std |
| | of | Average | Dev | of | Average | Dev |
| | measurements | (nm) | (nm) | measurements | (nm) | (nm) |
| Single | 50 | 10.2 | 0.9 | 50 | 10.5 | 0.9 |
| Tube | | | | | | |
| Diameter | | | | | | |
| Double | 20 | 15.8 | 1.3 | 20 | 15.6 | 0.7 |
| Tube | | | | | | |
| Diameter | | | | | | |
| Inner | 10 | 48.7 | 1.4 | 20 | 51.1 | 3.7 |
| Diameter | | | | | | |
| of | | | | | | |
| complete | | | | | | |
| tube | | | | | | |
| assemblies | | | | | | |

According to this model, bundlemers align side-side to form the cross-section of a nanotube with a wall thickness of 2 nm, an outer diameter of 10 nm, and an inner diameter of 5-6 nm. Given the very small diameter of the tube, the contrast of the outer wall in cryoTEM does not accurately reflect the true wall thickness due to the limited number of bundlemers forming the complete tube circumference. These bundlemers likely align end-to-end with slight kinks or twists, contributing to the curvature, while side-to-side interactions further stabilize these nanotube assemblies. Such offset interactions could arise from a complex combination of multiple side-chain interactions occurring around bundlemer peripheries both vertically and horizontally. Minimizing hydrophobic interactions with the aqueous environment could facilitate the formation of a second connected nanotube sharing common bundlemers, as depicted in FIG. 19. However, TEM observations indicate few structures containing more than two overlapping nanotubes. This limitation is likely from angular alignment constraints creating a frustrated assembly with limited stability when additional nanotubes are added.

### Example 2 Conclusions

Through the incorporation of both non-natural and natural amino acids, the versatility of computationally designed peptide bundlemers to self-assemble into distinct porous lattice structures in aqueous solution via hydrophobic interactions was demonstrated. This example highlights the robustness of the computationally optimized coiled-coil peptide tetramer, which readily accommodated modifications in the 13^{th} and 19^{th} positions, resulting in an expanded library of hydrophobic functional side chains. Although all modifications were introduced at identical peptide sequence positions, subtle variation in hydrophobic side chain size, length, and chemical properties resulted in the formation of distinctly different porous lattices compared to previously reported alloc-modified bundlemer lattices. The non-natural amino acid side chains (alkyne and furan) each assembled into different lattice structures characterized by distinctly different structure factor signatures, attributable to discrete angular orientations of the hydrophobic group and intra- and interbundle stacking interactions. Moreover, despite having different side chain structures, the alkyne-, phenylalanine-, and tyrosine-functionalized bundlemers all formed the same porous lattice structure as confirmed by TEM/cryoTEM and SAXS. In contrast, the furan- and tryptophan-based bundlemers exhibited unique self-assembly behavior. Tryptophan induced the formation of curved nanotube-like structures with specific curvature, whereas furan modified bundlemers led to a potentially polymorphic lattice that complicated definitive characterization. Both cases highlight how shorter or sterically restricted hydrophobic side chains induce additional interactions beyond simple hydrophobic stacking for lattice stabilization, revealing the subtle complexities in these systems. Overall, the sequence-specific modularity and tunability of peptide bundlemer lattice pore size and overall lattice morphology was affected by strategic side-chain selection. The chemically versatile functional groups introduced here offer opportunities for further conjugation via click-chemistry approaches, thus providing avenues to chemically stabilize these self-assembled nanostructures for future applications.

### Example 3: Single-Charge Peptide Bundlemer Networks Driven by Electrostatic and Hydrophobic Interactions

### Methods of Example 3

### Peptide Synthesis

Peptides were synthesized via microwave-assisted solid-phase peptide synthesis (SPPS) using a CEM Liberty Blue. Rink amide resin with loadings varying from 0.3-0.6 mmol/g was used as the solid support. Fmoc amino acids were prepared at 0.2 M in dimethyl formamide (DMF), and coupling agents oxyma and diisopropylcarbodiimide were prepared at 1M. Piperidine was prepared at 20% v/v in dimethyl formamide for Fmoc deprotection. Couplings were conducted at 90° C. for 4 minutes.

### Peptide Cleavage

After synthesis, the resin was triple-washed with DMF, methanol (MeOH), and dichloromethane (DCM). Resin was dried under nitrogen gas for 15 minutes before adding the cleavage cocktail. Cocktail consisted of 90 v/v % trifluoroacetic acid (TFA), 5v/v % triisopropylsilane (TIPS), and 5 v/v % Milli-Q water. Phenol and dithiothreitol (DTT) were added at a 50 mg/mL concentration. For a 0.25 mmol scale synthesis, 20 mL of cleavage cocktail was used. After adding the cocktail, the resin was shaken using a wrist-action shaker for 2 hours. After cleavage, TFA was precipitated dropwise in chilled diethyl ether and centrifuged at 4,000 rpm for 5 minutes. Supernatant was discarded and the pellet was washed twice with chilled diethyl ether. After the second wash, pellets were dried.

### Peptide Purification

Once dried, pellets were dissolved in Milli-Q water & purified via reverse-phase high-performance liquid chromatography (HPLC). Purification was conducted on a Waters 2535 Quaternary Gradient Module HPLC using a mobile phase of water (0.1 v/v % trifluoroacetic acid) and acetonitrile (0.1 v/v % trifluoroacetic acid). A C-18 column was used. Purification was conducted with a 1%/min concentration gradient from 20% to 60% acetonitrile. The molecular weight of HPLC fractions was confirmed using ultraperformance liquid chromatography-electrospray ionization-tandem mass spectrometry (UPLC/ESI-MS). Pure fractions were frozen in liquid nitrogen and lyophilized for 3 days. Lyophilized peptide was stored at −20° C.

### Circular Dichroism (CD) Spectroscopy

Experiments were conducted on a JASCO 1500 CD spectrometer. Peptide solutions were prepared at 0.1 mM in pure Milli-Q water. Quartz cuvettes with a pathlength of 1 mm were used. Experiments were conducted at 20° C. Measurements were collected from 250 nm to 190 nm wavelength, averaging 3 accumulations.

### Polarized Optical Microscopy (POM)

Samples were prepped in pure Milli-Q water at 5 w/w %, 10 w/w %, 15 w/w %, and 20 w/w %. The samples were aged for 3 days before imaging. Samples were prepped using a positive displacement pipette to dispense 5 μL of sample on a clean glass microscope slide. A glass coverslip was placed on the sample. Imaging was conducted on a Nikon Eclipse LV100 POL microscope equipped with a Nikon DS-Ri1 camera. Images were captured using NIS-Elements software and processed using Adobe Photoshop.

### Transmission Electron Microscopy (TEM)

TEM grids were purchased from Electron Microscopy Sciences. Ultrathin carbon with a mesh size of 200 or 400 were used. Grids were glow-discharged for 30 seconds using Pelco easiGlow™ glow discharge cleaning system. Samples were diluted to approximately 1-5 mM for each grid without mixing and deposited directly on the grids. 5 μL of peptide solution was pipetted and allowed to sit for 60 seconds before being wicked away using a KimWipe. 5 μL of 2 w/v % phosphotungstic acid (PTA) stain was added and allowed to sit for 50 seconds before being wicked away. Grids were left to dry in ambient conditions for at least 10 minutes before imaging. Samples were imaged on an FEI TALOS F200C TEM using an accelerating voltage of 200 kV. Image processing was conducted in Adobe Photoshop.

### Cryo-Transmission Electron Microscopy (TEM)

Lacey carbon type C TEM grids were purchased from Ted Pella, Inc. Grids were glow discharged for 30 seconds using Pelco easiGlow™ glow discharge cleaning system. No dilutions were conducted before sample preparation. Samples were prepared using a Vitrobot with a 100% humidity chamber. Samples were imaged on a FEI TALOS F200C TEM using an accelerating voltage of 200 kV. Image processing was conducted in Adobe Photoshop.

### Small Angle X-Ray Scattering (SAXS)

Quartz capillaries with a thickness of 0.01 mm and an outer diameter of 1.5 mm were used (Charles Supper Company). Sample concentration varied for experiments. Experiments were conducted using two different sources: NSLS-II 16-ID (LiX) at Brookhaven National Laboratory (BNL) and Xeuss Xenocs 2.0. Data from Brookhaven National Laboratory was obtained at NSLS-II 16-ID (LiX) beamline with a beam energy of 15.1 keV (0.82 Å). Exposure time was 0.5 s for each sample. Data obtained on a Xeuss Xenocs 2.0 used SAXS1 and standard exposure with exposure times ranging from 2-6 hours per sample. The beam energy was 8 keV (1.55 Å).

### Liquid Crystal to Lattice Transition

Solutions were prepped directly in Milli-Q water. Liquid crystal phase was confirmed via POM. A transition from the liquid crystal state to the lattice conformation was induced by adding a 5M NaCl solution to achieve a final concentration of 1M NaCl. Samples were manually mixed and placed in a bath sonicator for 2 hours. Bath sonication was conducted for multiple days (2 hours per day). Samples were parafilmed to prevent evaporation. Lattice formation was confirmed via SAXS.

### Lattice to Liquid Crystal Transition

Solutions were prepped at 10 w/w % in 0.5M NaCl. Lattice packing was confirmed using SAXS. A transition from the lattice to liquid crystal state was induced via a solvent exchange combined with slow evaporation and sonication. First, 50 μL of peptide solution was added to a centrifugal concentrator (Amicon® Ultra Centrifugal Filter, 0.5 mL, regenerated cellulose membrane, 3 kDa MWCO). Solutions were diluted with 200 μL of pure Milli-Q water and mixed. Filter units were then placed in a microcentrifuge and spun at 14,100 rcf for 30 minutes. After centrifugation, approximately 50 μL remains in the filter (5× concentrated). 200 μL of Milli-Q water was added, mixed, and centrifuged. This wash step was repeated 3 times. After proper salt removal, solutions were removed from the filter unit and transferred to microcentrifuge tubes. To achieve slow evaporation, samples were placed on a thermomixer set at 45° C. and 500 rpms. Samples were heated for 3-4 hours to achieve approximately 50% solution evaporation. After evaporation, samples were parafilmed and placed in a bath sonicator heated to 40° C. for 3 hours. After bath sonication, samples were aged at room temperature for 3 days before POM imaging.

### Lattice Structure Modelling

The lattice structure determination followed the previously published protocol using coarse-grained (CG) modeling and structure factor calculation along with machine learning optimization techniques (see Shi, Y. et al., Ordered assemblies of peptide nanoparticles with only positive charge. Nat Commun 2024, 15 (1), 10057; and McCahill, A. L. et al., Peptide Bundlemer Networks or Lattices: Controlling Cross-Linking and Self-Assembly Using Protein-like Display of Chemistry. ACS Nano 2024, 18 (37), 25695-25707). Based on the observation from TEM and SAXS, for the SC+6_2A, the initial construct of the unit cell, serving as the building block of our models, was posited to consist of two bundlemers that were orientated orthogonally with respect to each other. The inter distance among the bundlemer within the same unit cell (x_{d}, y_{d}, z_{d}) and the dimensions of the unit cell (xᵤ, yᵤ, zᵤ), were optimized via Bayesian optimization based on the calculated structure factors from the model and SAXS results achieved experimentally. During the optimization, the positions of the peaks achieved from the calculated structure factors via modelling were matched with those experimentally observed structure factors from SAXS. Here, the 4 major peaks from 3^{rd} to 6^{th} in SAXS were used for the optimization. The parameter values with lowest mean absolute error (0.02 nm) were (in units of nm): x_{d}=0.28, y_{d}=2.49, z_{d}=0.37, xᵤ=4.0, yᵤ=4.98, zᵤ=3.98. The detailed information about the modeling was provided in previous work (see Shi, Y. et al., Ordered assemblies of peptide nanoparticles with only positive charge. Nat Commun 2024, 15 (1), 10057).

For SC+8_2A, the assembled structure closely resembles the FCC truss lattice, thus prompting the modeling of the relative orientations of the CG bundlemers similarly. A small portion of lattice consisting of 6 CG bundlemers was created and grouped into three pairs. Within each pair, the two CG bundlemers colinearly align end-to-end throughout the optimization process. The refinement parameter, L, is the distance between the center of mass (COM) of the two CG bundlemers in each pair. Next, one pair along the y axis was fixed and the other two pairs were generated so as to match the relative strut orientations within the FCC truss lattice. The variable lattice parameters were L and the Cartesian coordinates of each of the three bundle pairs in the lattice repeat unit (x₁, y₁, z₁, x₂, y₂, z₂, x₃, y₃, z₃). The first 5 major peaks in SAXS were used for the optimization. The parameter values with lowest mean absolute error (0.07 nm) were L=4.98, x₁=−3.65, y₁=−0.42, z₁=2.29, x₂=−2.47, y₂=1.48, z₂=3.30, x₃=−3.36, y₃=2.81, z₃=−1.94. The detailed information about the modeling was provided in previous work (see McCahill, A. L. et al., Peptide Bundlemer Networks or Lattices: Controlling Cross-Linking and Self-Assembly Using Protein-like Display of Chemistry. ACS Nano 2024, 18 (37), 25695-25707).

### Example 3 Experiments and Results

### Experiment 1: Single Charge Bundlemer Design

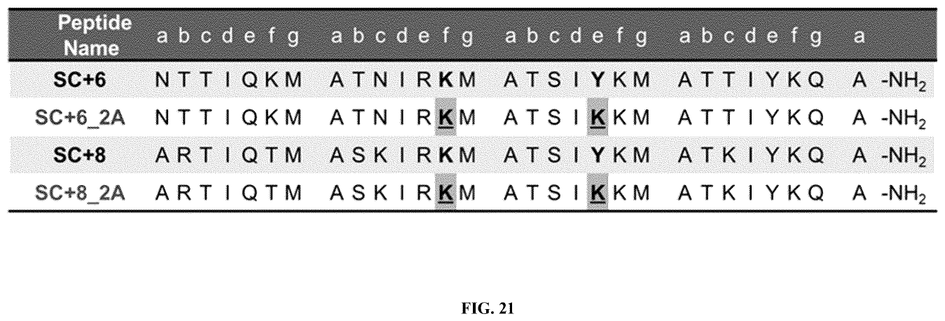

Two single charge (SC) type bundlemer sequences, SC+6 (SEQ. ID. NO. 15) and SC+8 (SEQ. ID. NO. 16), were chosen as parent sequences for selective modification due to their ability to form liquid crystals in aqueous solution. With each peptide forming a homotetrameric, antiparallel coiled-coil bundlemer particle, the putative charge on the final parent particles is +24 and +32, respectively. These sequences have been selectively modified with an alloc protected lysine in the 13^{th} and 19^{th} amino acid positions (see FIG. 21), mimicking the modifications previously made to the mixed charge (MC) 4B+4 sequence that drove nanoporous lattice self-assembly via hydrophobic interactions in Example 1. The alloc modification produces SC+6_2A and SC+8_2A with a +5 or +7 charge per peptide (+20 or +28 per bundlemer particle), respectively, where the net charge of the two parent peptides was reduced by one positive residue, as these sites were originally occupied by a lysine (positive) and a tyrosine (non-charged), respectively. Despite making these mutations to the existing peptide sequences, the coiled-coil structure is conserved as demonstrated by the circular dichroism spectroscopy with characteristic α-helical minima at 208 and 222 nm. The ability to selectively modify these SC sequences with new chemical functionality highlights the robustness of the hydrophobic core from the computational design of bundlemer sequences.

### Experiment 2: Liquid Crystal Behavior Assessment

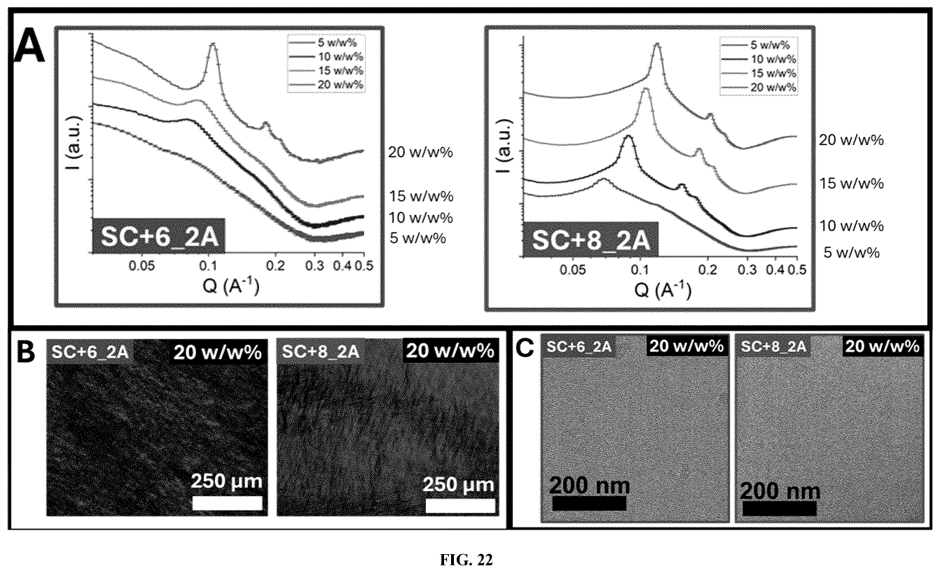

After parent sequence modification, liquid crystal behavior in water was evaluated to assess whether the alloc modifications disrupted bundlemer end-to-end stacking and liquid crystal assembly. The electrostatic interactions and end-to-end stacking were hypothesized to still dominate in salt-free aqueous solution. Lateral electrostatic repulsion should disallow hydrophobic interactions between alloc side chains of lateral neighboring particles since they require close proximity to stabilize and form. FIG. 22 shows the small-angle x-ray scattering (SAXS) data for SC+8_2A and SC+6_2A at 5, 10, 15, and 20 w/w % in Milli-Q water. Each curve for the SC+8_2A exhibits clear diffraction peaks with notable peak ratios of q*, \sqrt{3}q*, \sqrt{4}q*, characteristic of a two-dimensional hexagonal packing of long bundlemer chains stacked in an end-to-end fashion. The observed q value of the diffraction peaks between samples is dependent on the concentration of the peptide in solution with the higher concentration of peptide resulting in a closer packing of bundlemer chains in their hexagonal lattice, consequently shifting the peaks towards higher q. Similar behavior was observed for SC+6_2A with hexagonal peaks not appearing till approximately 10 w/w %, meaning the observed critical LC concentration is higher for SC+6_2A as compared to SC+8_2A. Additionally, the hexagonal diffraction peaks are broader for SC+6_2A, likely due to the lower electrostatic interaction between chains of SC+6_2A that lowers the interchain repulsion and hexagonal ordering with this bundlemer building block.

LC behavior for both samples was further validated using polarized optical microscopy (POM) (see FIG. 22). For SC+8_2A, birefringence can be observed in all samples, highlighting the liquid crystallinity of these samples across a range of concentrations, with a critical concentration for LC formation below 5 w/w %. For SC+6_2A, there was no observed birefringence at 5 w/w %, further supporting the absence of an LC at the lowest concentration. For the remaining concentrations, birefringence can be observed in all images. CryoTEM images of samples at 20 w/w % are shown in FIG. 22 revealing highly aligned, 1D, physically assembled bundlemer chains. The POM, SAXS, and cryoTEM results demonstrate that the LC behavior was conserved for both alloc-modified sequences. The SC+6 unmodified parent sequence (i.e., without added alloc groups) displayed a similar critical concentration for LC formation, ranging between 5% and 10% w/w. For the SC+8 unmodified sequence, the previous critical concentration was observed between 5 and 10 w/w %, which is higher than the observed critical concentration of SC+8_2A, which is below 5 w/w %. Both alloc-modified sequences exhibit similar or lower critical LC concentrations, further supporting the claim that these modifications do not detrimentally impact LC formation in solution.

### Experiment 3: Lattice Nanostructure Formation Assessment

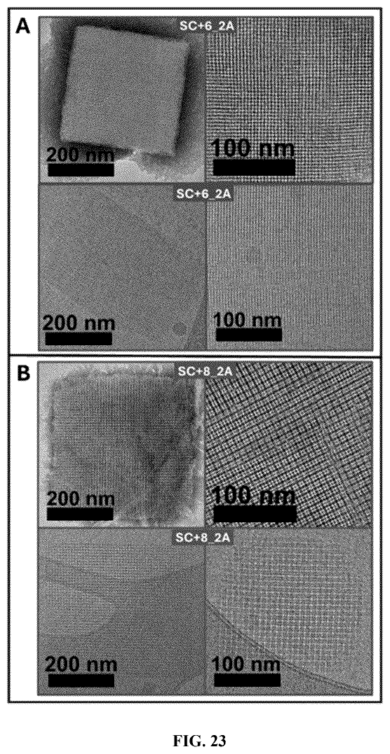

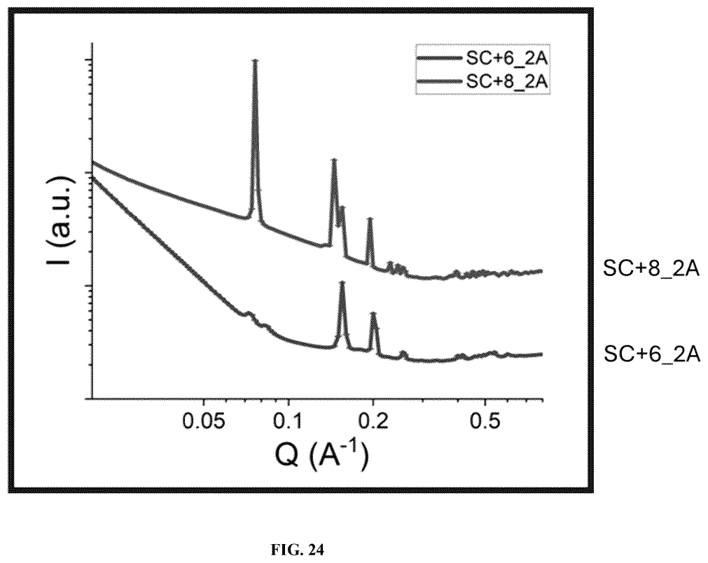

In bundlemer solutions with no salt, the alloc-modified SC+6_2A and SC+8_2A samples both robustly stack in an end-to-end fashion to form chains that are electrostatically repulsive laterally and form hexagonal columnar LC phases. However, by screening electrostatic interactions with added salt, the repulsive forces stabilizing hexagonal columnar LC phase will be eliminated, thus allowing the hydrophobic alloc side chain interactions to affect interparticle assembly. To test this hypothesis, samples were prepared in either 500 mM or 1 M NaCl aqueous solutions. Both alloc-modified sequences immediately became turbid on dissolution in either salt solution. FIG. 23 depicts the TEM and cryoTEM results for both modified sequences in 1M NaCl salt solution. In both samples, well defined nanosheets are easily observed with slightly different lattice projections. SAXS data is shown in FIG. 24, demonstrating differences in the structure factor signature for both sequences. Despite having the same site-specific alloc modification, it appears that SC+6_2A and SC+8_2A form somewhat different nanoporous lattice structures in solution, as indicated by their different observed lattice projections and differences in structure factor SAXS signature. Overall, differences in lattice packing of the SC+6_2A and SC+8_2A particles are likely caused by their subtle differences in their amino acid composition with most of the differences in composition present in the first two heptads of the two sequences.

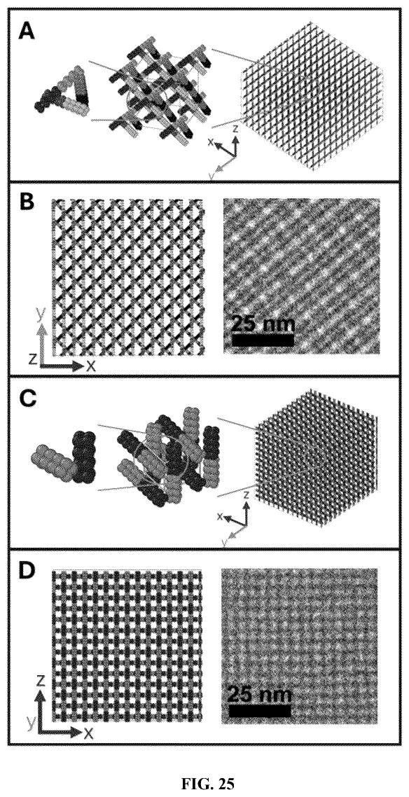

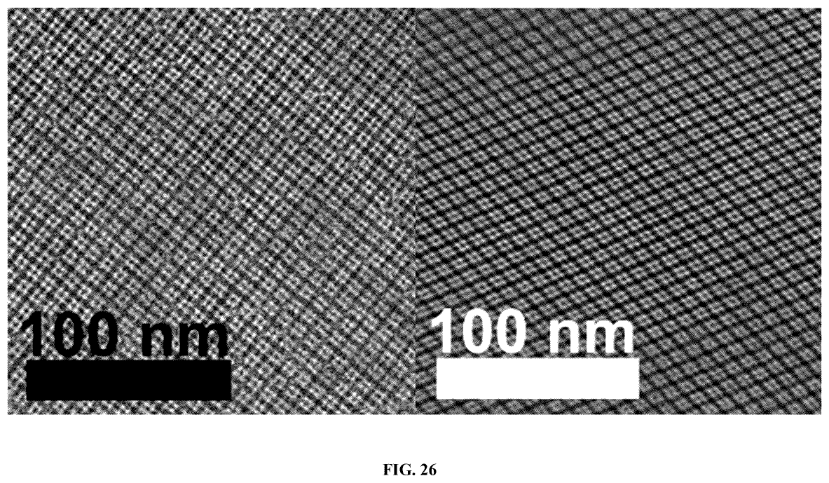

To study the differences in nanostructure between the two peptide sequences, a lattice model was developed for SC+6_2A and SC+8_2A via a machine learning optimization approach to match experimental structure factor data to a coarse-grained bundlemer lattice model. The determined model for SC+8_2A can be found in FIG. 25, with projection comparisons with cryoTEM and the model. Uniquely, SC+8_2A has an almost identical SAXS signature to the previously studied 4B+4_2A lattices with the mixed charged 4B+4 parent sequence, indicating that these lattices must have similar, or the same, interbundlemer packing underlying the respective lattices. This observation of similar lattice structures for a mixed charge and single charge sequence is critical because these sequences differ significantly in their amino acid composition. Aside from an identical hydrophobic core (11 residues) and the alloc-protected lysines (2 residues) in positions 13/19, these sequences share only 4 surface residues in common (out of 16). FIG. 26 includes two projections from negative stain TEM, one from SC+8_2A and one from 4B+4_2A. These projections highlight the similarities in nanostructure between the two lattices. Despite the differences in sequence, when electrostatic interactions are screened, the SC+8_2A sequence behaves similarly to 4B+4_2A and forms an FCC truss-like lattice, highlighting the importance of the display of the allocs in the 13/19 positions and the domination of the hydrophobic interactions in the formation of identical lattice structure.

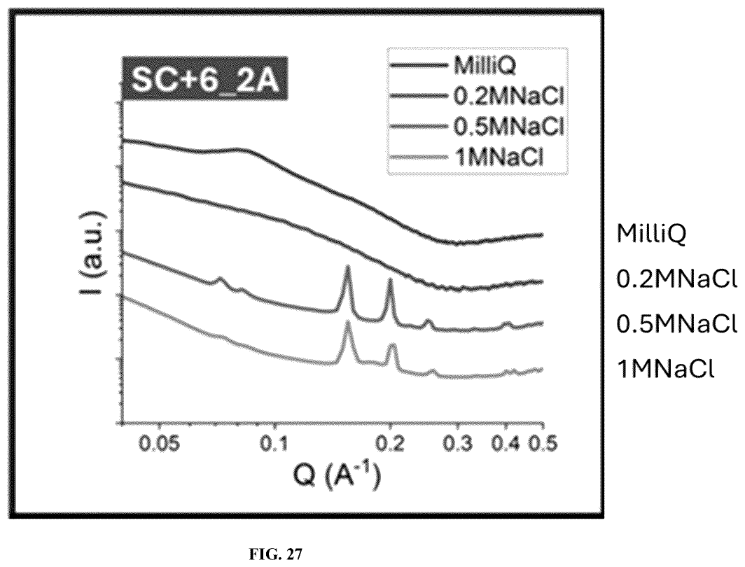

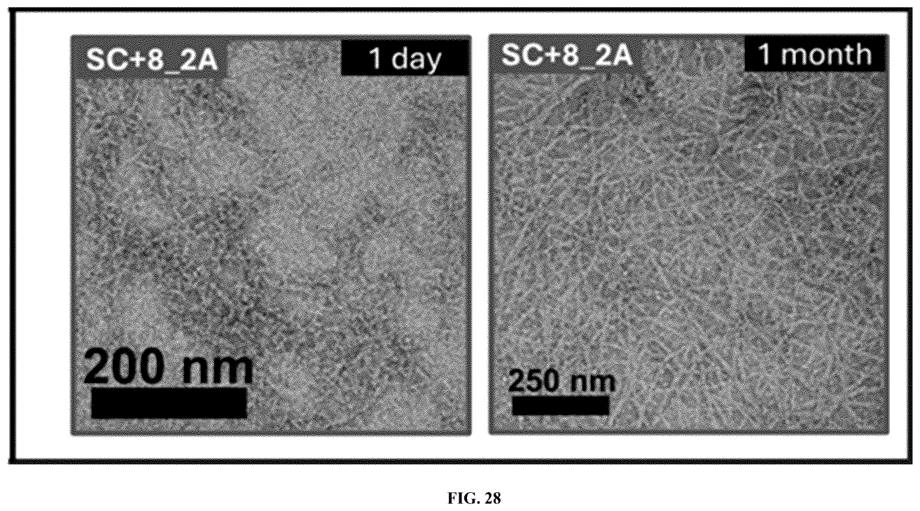

For SC+6_2A, several major peaks from the structure factor signature were matched to the determined model shown in FIG. 25. The broad, weak peaks at low Q were excluded from the model determination, because they were not observed for all salt concentration conditions, as shown in FIG. 27, while the strong diffraction at higher q values was reproducible across different salt conditions. The emergence of these broad, weak peaks at lower q values could be attributed to a sheet thickness/sheet stacking with the peaks observed at q=0.072 Å⁻¹ (d=8.7 nm) and q=0.083 Å⁻¹ (d=7.6 nm), which correspond to approximately two bundlemer particles stacked end-to-end or four bundlemer particles stacked side-to-side. For SC+6_2A, the particles assemble perpendicular to each other, resulting in a square lattice morphology with close bundlemer packing. As observed in other bundlemer lattice models, there is a coupling of end-to-end stacking of the particles and side-to-side hydrophobic alloc interactions. Experimental cryoTEM was matched to the computational model derived from structure factor signature in SAXS, and projections are shown in FIG. 28. The SC+6_2A lattice is orthorhombic-like with 4-fold-like projections observed in the TEM.

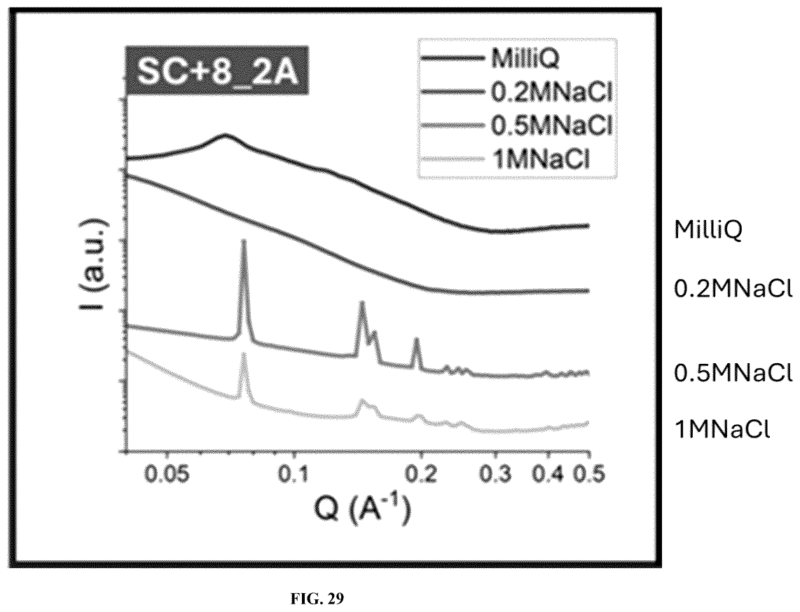

Since electrostatic interactions strongly impact liquid crystal formation, salt screening plays a vital role in the emergence of the hydrophobic lattice structures. To probe the effect of salt concentration on self-assembly and to determine the most appropritae salt concentration for lattice formation, sequences were prepped and suspended directly in salt solutions with concentrations of 0 M, 0.2 M, 0.5 M, and 1 M NaCl. Peptide sequences were studied above their critical liquid crystal concentration, 5 w/w % and 10 w/w % for SC+8_2A and SC+6_2A, respectively. The nanostructure was evaluated using SAXS to track the structure factor signature of the liquid crystal vs. lattice packings. FIG. 27 and FIG. 29 include the SAXS data for SC+6_2A and SC+8_2A. At 0 M NaCl (pure Milli-Q water), both SC+6_2A and SC+8_2A exhibited LC behavior with clear hexagonal packing. At 0.2 M NaCl, there was enough charge screening from the sodium chloride to obviate the liquid crystallinity of the solution. However, no distinct lattice diffraction was observed. At 0.5 M NaCl, structure factor diffraction peaks were observed, resulting from the formation of lattice particles in solution. Finally, at 1 M NaCl, lattice peaks were still present but less prominent.

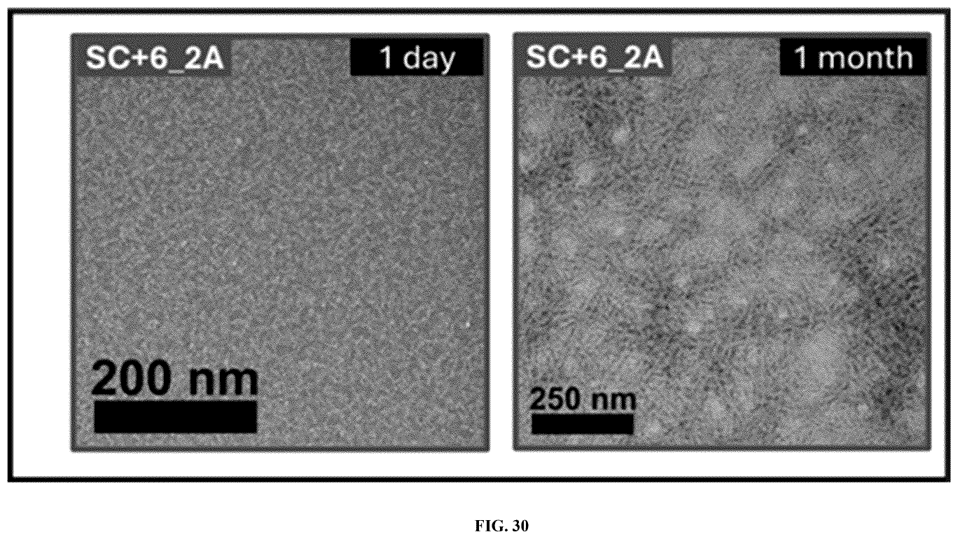

Overall, these data provided an optimized salt concentration target centered around 0.5 M NaCl for the formation of lattice particles for the peptides at these concentrations. Liquid crystals and lattices could thus be targeted with salt to effectively disrupt both phases. This allowed the nanoparticles to exist in solution with no regular ordered structure. The 0.2 M NaCl solutions were studied over time using TEM to track the possibility of assembly into either liquid crystal or lattice domains when aged. FIG. 28 and FIG. 30 show the negative-stain TEM of SC+6_2A and SC+8_2A, with samples aged 1 day (left) and 1 month (right), respectively. As seen in the TEM image after just one day, both sequences exhibited mostly nonspecific aggregation, with a small number of nanofibrils forming through end-to-end assembly of the particles in solution. After 1 month, more nanofibrous structures were observed. Therefore, sample aging allowed the particles to assemble into nanofibrils via end-to-end stacking; however, these fibrils can ultimately not interact laterally to form large liquid crystalline domains or nanoporous lattice particles via side-to-side interactions because of electrostatic screening. These results demonstrated that the polymorphic nature of the designed nanoparticle, which can assemble into two entirely different ordered phases (liquid crystal and lattice) and a relatively disordered phase of non-interacting nanofibrils, could be readily manipulated by the level of salt screening.

### Experiment 4: LC to Lattice Structure Transition Evaluation

The potential for a transition between the two ordered phases was investigated to probe the responsiveness of the ordered structures to changes in salt concentration in solution. In addition, impacts of an order-order assembly pathway on resultant nanostructures could be directly observed. This transition proved challenging due to the highly stable nature/high viscosity of the LC phases and the differences in bundlemer particle orientation within the LC phases vs the porous lattices. The liquid crystal domains existed as hexagonal columnar phases in which the individual bundlemer particles stack in an end-to-end fashion to form 1D chain structures, with chains aligned in a parallel orientation. On the other hand, the nanoporous lattices exhibited a truss-like structure in which end-to-end bundlemer chains structures interacted with neighboring chains at sharp, sometimes perpendicular angles quite unlike the parallel chain alignment in the LCs. Therefore, the electrostatic interactions stabilizing the ordered liquid crystal domains not only have to be weakened or broken by electrostatice screening, but the bundlemer particles must also significantly change their orientation relative to each other in a concentrated solution state to form the ordered lattice structure.

The transition from pure water to concentrated salt was first evaluated. The transition between the liquid crystal (LC) state and the lattice state (and vice versa) can be observed through changes in the turbidity and viscosity of the solution, which are easily visible. The liquid crystal is optically transparent and highly viscous, while the lattice particles form a turbid, more low viscosity suspension. SC+8_2A was the focus of these experiments due to its resemblance to the mixed charge lattices that were previously characterized in Example 1. Initially, SC+8_2A was solubilized directly in Milli-Q water and dialyzed against a concentrated salt solution for several days, with periodic solvent exchange. This dialysis approach did not yield significant results for transitioning from a liquid crystal to a lattice phase. Instead, results were like those observed in 0.2 M NaCl solutions, in which disordered nanofibrils were most observed. The addition of salt only disrupted side-to-side interactions in the liquid crystal chains rather than resulting in a complete conversion to a lattice assembly. These results suggested that the diffusion of salt alone was insufficient to disrupt the liquid crystal phase sufficiently to transition to the ordered lattice phase.

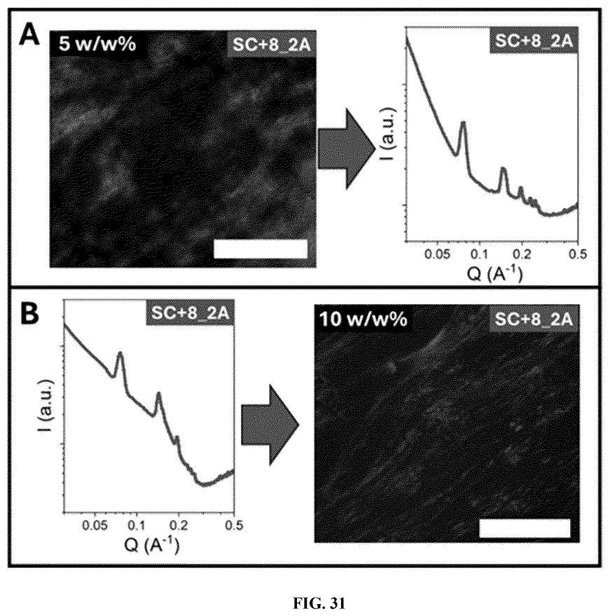

Partial charge screening could promote interaction among the hydrophobic side chains around the center of the individual bundlemers and ultimately reinforce the bundlemers' parallel orientation originally formed in the liquid crystal phase. Because dialysis did not induce a transition into an ordered lattice, a second approach involving the quenching of the liquid crystals with concentrated salt and subsequent sonication was explored. SC+8_2A was solubilized in Milli-Q water at a 5% w/w concentration, and the presence of liquid crystal domains was confirmed via POM (see FIG. 31). A small volume of concentrated salt was pipetted into the solution and mixed vigorously. The solutions were manually mixed and bath sonicated to ensure homogeneous salt distribution and to break up ordered structures and allow for the rearrangement of bundlemers into an ordered lattice structure. This approach successfully transitioned from a liquid crystal to a lattice assembly. FIG. 31 also includes the POM of the liquid crystal solution before salt/mixing treatment and the SAXS of the suspension after mixing with salt and a partial conversion to lattice particles. While salt diffusion alone was insufficient to induce the transition from liquid crystal to lattice, sonication proved to be an excellent tool for weakening interparticle interactions in the liquid crystal state and affording the formation of the nanoporous lattice particles.

Transitioning from the lattice state to the liquid crystal phase also proved challenging. Initial attempts involved preparing SC+8_2A suspensions directly in 0.5 M NaCl and then using dialysis to remove NaCl. Like the previous pathway, diffusion alone appeared insufficient to transition between the lattice structure and LC phases because of the stability of the already formed lattice and extensive hydrophobic interactions. As demonstrated previously, the final concentration of salt can influence the formation of liquid crystals, and, perhaps, remaining amounts of salt also could prevent the formation of the liquid crystal phase from the lattice structure. To overcome these challenges, a solvent exchange approach was explored.

The bundlemers were prepared at 10 w/w % in 0.5 M NaCl, and lattice packing was confirmed via SAXS (see FIG. 31). The solutions were then diluted with pure Milli-Q water. After dilution, the samples were loaded into a centrifugal filter unit with an appropriate molecular weight cutoff to ensure the retention of the bundlemer structures and the removal of salt. Samples were centrifuged at high speeds to allow the solvent to pass through the filters while retaining the peptide. This wash approach was repeated several times to remove salt while maintaining the peptide concentration in solution. After centrifugal filtration, samples were concentrated via controlled evaporation and bath sonicated. This step increased concentration and helped break up non-specific aggregation during the previous processing steps. POM was conducted to evaluate the presence of the liquid crystal phase. The SAXS data before salt removal and the POM data after salt removal are shown in FIG. 31. The SAXS shows that the structure factor signature is present for SC+8_2A lattice particles. After salt removal, the POM shows regions of strong birefringence, supporting the formation of the liquid crystal phase via this method. These results demonstrate the stability of the LC phases and the nanoporous lattices and highlight the need for significant physical agitation and solvent processing to weaken interparticle interactions to achieve a successful order-order transition between phases.

### Conclusions of Example 3

A single peptide coiled-coil design was shown to form two distinct, ordered phases by simple alteration of solution conditions to manipulate electrostatic screening between particles. Single-charge type (SC) bundlemer coiled-coil nanoparticles were successfully modified with hydrophobic side chains to create polymorphic peptide particles capable of self-assembly into two different ordered structures: hexagonal columnar liquid crystals in pure water and nanoporous lattices in concentrated salt solution. Uniquely, the bundlemers existed in the same folded coiled-coil state for both phases, but through control of the surface interactions between particles, they can adopt two different interparticle configurations. These results demonstrate the robustness of the LC phase of these SC sequences in pure water and highlight their ability to be selectively modified with hydrophobic interactions on their particle surface without disrupting their LC assembly. In contrast, after salt addition and consequent electrostatic screening, the alloc-driven hydrophobic self-assembly occured over a range of bundlemer parent sequences with different amino acid compositions to form nanoporous lattices. Despite the differences in the peptide sequences, the alloc modification for both SC bundlemers and the \ mixed charge type bundlemer sequences of Example 1 resulted in similar lattice formation through hydrophobic interactions, highlighting the importance of the display of functional groups on the bundlemer particle periphery and the specificity of their interactions that drive self-assembly. The functional design of these peptide particles has enabled tunable control over the assembled nanostructure, with potential in the bottom-up fabrication of peptide-based materials. Using a single molecule capable of adopting two distinctly different ordered phases from the same self-assembled coiled-coil building block has potential applications in templating, sensing, or smart materials. The ability to selectively modify individual side chains in the bundlemer particles provides for amino acid side chain resolution and protein-like specificity in the design of interparticle interactions and the formation of targeted nanostructures

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1. A tetrameric coiled-coil bundlemer comprising:
four peptides, each peptide possessing a first amino acid sequence (1^{st}), a second amino acid sequence (2^{nd}), a third amino acid sequence (3^{rd}) and a fourth amino acid sequence (4^{th}),
wherein the 1^{st}, 2^{nd}, 3^{rd} and 4^{th} amino acid sequence possess a heptadic peptide sequence abcdefg and are arranged as follows: 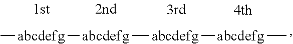
wherein:
(i) the 1^{st}, 2^{nd}, 3^{rd} and 4^{th} amino acid sequences together form an alpha-helical structure;
(ii) the a, d, and g positions of each of the four peptides form a hydrophobic core of the tetrameric coiled-coil bundlemer structure, wherein:
the a position of the 1^{st} amino acid sequence is aspartic acid (D) and the a position of the 2^{nd}, 3^{rd} and 4^{th} amino acid sequences is alanine (A),
the d position of the 1^{st}, 2^{nd}, 3^{rd} and 4^{th} amino acid sequences is isoleucine (I), and
the g position of the 1^{st}, 2^{nd}, and 3^{rd} amino acid sequences are methionine (M) and the g position of the 4^{th} amino acid sequences is either methionine (M) or glutamic acid (E);
(iii) each b, c, e, and f is an amino acid exposed on a surface of the bundlemer structure; and
(iv) at least one of the b, c, e, and f positions of the 2^{nd} and/or 3^{rd} amino acid sequence is selected from the group consisting of a modified lysine amino acid possessing an allyloxy carbonyl moiety, a modified lysine amino acid possessing an alkyne moiety, a modified lysine amino acid possessing a furan moiety, phenylalanine (F), tyrosine (Y) and tryptophan (W).

2. The bundlemer of claim 1, wherein each constituent peptide has a charge ranging from −8 to +8.

3. The bundlemer of claim 1, wherein at least one of the b, c, e, and f positions of the 2^{nd} and/or 3^{rd} amino acid sequence is a modified lysine amino acid possessing an allyloxy carbonyl moiety.

4. The bundlemer of claim 3, wherein the bundlemer possesses a cysteine (C) (i) before the a position of the 1^{st} amino acid sequence and, optionally, after the g position of the 4^{th} amino acid sequence; and
wherein the f position of the 2^{nd} amino acid sequence and, optionally, the e position of 3^{rd} amino acid sequence are a modified lysine amino acid possessing an allyloxy carbonyl moiety.

5. The bundlemer of claim 1, wherein the b position of the 1^{st} amino acid sequence is a modified lysine amino acid possessing an allyloxy carbonyl moiety, the f position of the 4^{th} amino acid sequence is cysteine, the e position of 3^{rd} amino acid sequence is a modified lysine amino acid possessing an allyloxy carbonyl moiety, and the 1^{st} acid sequence possess a cysteine before the a position.

6. The bundlemer of claim 1, wherein the f position of the 2^{nd} amino acid sequence and the e position of 3^{rd} amino acid sequence are a modified lysine amino acid possessing an allyloxy carbonyl moiety.

7. The bundlemer of claim 1, wherein the bundlemer has a diameter of about 2 nm and a length of about 4 nm.

8. The bundlemer of claim 1, wherein:
the f position of the 2^{nd} amino acid sequence and the e position of 3^{rd} amino acid sequence are a modified lysine amino acid possessing an alkyne moiety;
the f position of the 2^{nd} amino acid sequence and the e position of 3^{rd} amino acid sequence are phenylalanine; or
the f position of the 2^{nd} amino acid sequence and the e position of 3^{rd} amino acid sequence are tyrosine.

9. The bundlemer of claim 1, wherein the f position of the 2^{nd} amino acid sequence and the e position of 3^{rd} amino acid sequence are tryptophan.

10. The bundlemer of claim 1, wherein the f position of the 2^{nd} amino acid sequence and the e position of 3^{rd} amino acid sequence are a modified lysine amino acid possessing a furan moiety.

11. An amorphous network comprising more than one bundlemer of claim 5.

12. The amorphous network of claim 9, wherein the network possesses a storage modulus (G′) of about 50 kPa to about 2,300 kPa.

13. A lattice nanostructure comprising more than one bundlemer of claim 6.

14. The lattice nanostructure of claim 13, wherein the lattice nanostructure possesses an average pore size of about 5 to about 6 nm.

15. The lattice nanostructure of claim 13, wherein the lattice nanostructure possesses a truss-like face-centered cubic (FFC) lattice symmetry.

16. The lattice nanostructure of claim 13, wherein the lattice nanostructure is found within diamond shaped particle morphology.

17. The lattice nanostructure of claim 13, wherein the lattice nanostructure is found within rectangular shaped particle morphology.

18. A lattice nanostructure comprising more than one bundlemer of claim 8.

19. The lattice nanostructure of claim 18, wherein the lattice nanostructure possesses chains of end-to-end stacked bundlemer particles aligned on a square lattice.

20. The lattice nanostructure of claim 18, wherein the lattice nanostructure possesses an average pore size of about 2 to about 3 nm.

21. A rounded nanostructure comprising more than one bundlemer of claim 9.

22. The rounded nanostructure of claim 21, wherein the rounded nanostructure possesses an outer diameter of about 10 to about 13 nm, an inner diameter of about 5 to about 6 nm and/or an average thickness of about 2 nm.

23. A polymorphic lattice formed from more than one bundlemer of claim 10.

24. A tetrameric coiled-coil bundlemer comprising:
four peptides, each peptide possessing a first amino acid sequence (1^{st}), a second amino acid sequence (2^{nd}), a third amino acid sequence (3^{rd}) and a fourth amino acid sequence (4^{th}),
wherein the 1^{st}, 2^{nd}, 3^{rd} and 4^{th} amino acid sequence possess a heptadic peptide sequence abcdefg and are arranged as follows: 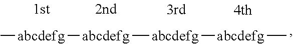
wherein:
(i) the 1^{st}, 2^{nd}, 3^{rd} and 4^{th} amino acid sequences together form an alpha-helical structure;
(ii) the a, d, and g positions form a hydrophobic core of the tetrameric coiled-coil bundlemer structure, wherein:
the a position of the 2^{nd}, 3^{rd} and 4^{th} amino acid sequences is alanine (A),
the d position of the 1^{st}, 2^{nd}, 3^{rd} and 4^{th} amino acid sequences is isoleucine (I), and
the g position of the 1^{st}, 2^{nd}, and 3^{rd} amino acid sequences is methionine (M);
(iii) each b, c, e, and f is an amino acid exposed on a surface of the bundlemer structure; and
(iv) at least one of the b, c, e, and f positions of the 2^{nd} and/or 3^{rd} amino acid sequence is selected from the group consisting of a modified lysine amino acid possessing an allyloxy carbonyl moiety, a modified lysine amino acid possessing an alkyne moiety, a modified lysine amino acid possessing a furan moiety, phenylalanine (F), tyrosine (Y) and tryptophan (W).

25. A lattice nanostructure comprising more than one bundlemer of claim 24.

26. The lattice nanostructure of claim 25, wherein the lattice nanostructure possesses a truss-like face-centered cubic (FFC) lattice symmetry.

27. A method of creating an amorphous network comprising:
(i) solubilizing more than one bundlemer of claim 5 in an aqueous solution;
(ii) adding at least one photoinitiator into the aqueous solution; and
(iii) irradiating the solution to promote thiol-ene click chemistry reactions between the allyloxy carbonyl moieties and cysteines of the bundlemers.

28. The method of claim 27, wherein the aqueous solution contains about 0.25 g/mL to about 0.75 g/mL of the bundlemers.

29. The method of claim 27, wherein the solution contains about 20 mM of the at least one photoinitiator after (ii).

30. A method of creating a lattice nanostructure comprising:
(i) solubilizing more than one bundlemer of claim 1 into an aqueous solution;
(ii) adding at least one photoinitiator into the aqueous solution; and
(iii) irradiating the solution to promote crosslinking reactions,
wherein the aqueous solution contains about 25 wt % to about 75 wt % of the bundlemer.

31. An article formed from either a lattice nanostructure or amorphous synthetic polymer network containing the bundlemer of claim 1, wherein the article is a macromolecular material selected from catalytic membranes, chiral compound separators, ion separators, barrier plastics, nanostructured films for membranes, fuel cells, batteries, chemical separators, water desalination devices, biomedical devices, drug delivery devices, smart coatings in implants and diagnostic equipment, and cell scaffolds for tissue engineering.


SEQUENCE LISTING (16 sequences)

SEQ ID NO: 1 - protein, 30 aa, synthetic construct
  SITE 14: note = "X" is a modified lysine amino acid possessing an allyloxy carbonyl moiety
CDKEIRRMAEKIRXMAERIKQMAEQIYKEA

SEQ ID NO: 2 - protein, 30 aa, synthetic construct
  SITE 14: note = "X" is a modified lysine amino acid possessing an allyloxy carbonyl moiety
  SITE 20: note = "X" is a modified lysine amino acid possessing an allyloxy carbonyl moiety
CDKEIRRMAEKIRXMAERIXQMAEQIYKEA

SEQ ID NO: 3 - protein, 29 aa, synthetic construct
  SITE 2: note = "X" is a modified lysine amino acid possessing an allyloxy carbonyl moiety
  SITE 19: note = "X" is a modified lysine amino acid possessing an allyloxy carbonyl moiety
DXEIRRMAEKIRKMAERIXQMAEQIYKEA

SEQ ID NO: 4 - protein, 31 aa, synthetic construct
  SITE 14: note = "X" is a modified lysine amino acid possessing an allyloxy carbonyl moiety
  SITE 20: note = "X" is a modified lysine amino acid possessing an allyloxy carbonyl moiety
CDKEIRRMAEKIRXMAERIXQMAEQIYCEAC

SEQ ID NO: 5 - protein, 30 aa, synthetic construct
  SITE 3: note = "X" is a modified lysine amino acid possessing an allyloxy carbonyl moiety
  SITE 20: note = "X" is a modified lysine amino acid possessing an allyloxy carbonyl moiety
CDXEIRRMAEKIRKMAERIXQMAEQIYCEA

SEQ ID NO: 6 - protein, 29 aa, synthetic construct
  SITE 13: note = "X" is a modified lysine amino acid possessing an allyloxy carbonyl moiety
  SITE 19: note = "X" is a modified lysine amino acid possessing an allyloxy carbonyl moiety
DKEIRRMAEEIRXMAERIXQMAEQIYKEA

SEQ ID NO: 7 - protein, 29 aa, synthetic construct
  SITE 13: note = "X" is a modified lysine amino acid possessing an alkyne moiety
  SITE 19: note = "X" is a modified lysine amino acid possessing an alkyne moiety
DKEIRRMAEEIRXMAERIXQMAEQIYKEA

SEQ ID NO: 8 - protein, 29 aa, synthetic construct
  SITE 13: note = "X" is a modified lysine amino acid possessing a furan moiety
  SITE 19: note = "X" is a modified lysine amino acid possessing a furan moiety
DKEIRRMAEEIRXMAERIXQMAEQIYKEA

SEQ ID NO: 9 - protein, 29 aa, synthetic construct
DKEIRRMAEEIRFMAERIFQMAEQIYKEA

SEQ ID NO: 10 - protein, 29 aa, synthetic construct
DKEIRRMAEEIRWMAERIWQMAEQIYKEA

SEQ ID NO: 11 - protein, 29 aa, synthetic construct
DKEIRRMAEEIRYMAERIYQMAEQIQKEA

SEQ ID NO: 12 - protein, 29 aa, synthetic construct
  SITE 13: note = "X" is a modified lysine amino acid possessing an allyloxy carbonyl moiety
  SITE 19: note = "X" is a modified lysine amino acid possessing an allyloxy carbonyl moiety
NTTIQKMATNIRXMATSIXKMATTIYKQA

SEQ ID NO: 13 - protein, 29 aa, synthetic construct
  SITE 13: note = "X" is a modified lysine amino acid possessing an allyloxy carbonyl moiety
  SITE 19: note = "X" is a modified lysine amino acid possessing an allyloxy carbonyl moiety
ARTIQTMASKIRXMATSIXKMATTIYKQA

SEQ ID NO: 14 - protein, 29 aa, synthetic construct
DKEIRRMAEKIRKMAERIKQMAEQIYKEA

SEQ ID NO: 15 - protein, 29 aa, synthetic construct
NTTIQKMATNIRKMATSIYKMATTIYKQA

SEQ ID NO: 16 - protein, 28 aa, synthetic construct
ARTIQTMASKIRKMATSIYKMATKIYKQ